(19) United States
(12) Patent Application Publication
(10) Pub. No.: US 2002/0038113 A1
(43) Pub. Date: Mar. 28, 2002

(54) Apparatus and method for artificial insemination and embryo transfer of animals

(76) Inventors: Dennis D. Gourley, Waukon, IA (US); Gregory R. Lear, Spencer, IA (US); Mariano M. Raigo, Waukon, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 09/906,330

(22) Filed: Jul. 16, 2001

Related U.S. Application Data
(60) Provisional application No. 60/218,451, filed on Jul. 14, 2000.

(51) Int. Cl. A61M 31/00

(52) U.S. Cl. 604/515; 604/275

(57) ABSTRACT

Apparatus and methods for artificial insemination and embryo transfer relative to livestock animals is disclosed. The invention utilizes an inner sheath/distal probe end combination to allow atraumatic navigation through the cervical anatomy of the animal to reach the uterus. The probe end both is configured to assist in the navigation through the cervical anatomy as well as is intentionally minimized in size relative to the cervical anatomy. Once navigation is completed, semen or embryos can be emplaced at the desired site.

### BACKGROUND OF THE INVENTION

1. Field of the Invention

The present invention relates to several aspects to artificial insemination of animals, and in another aspect to embryo transfer in animals.

2. Problems In The Art

Economics, efficiencies, and genetics all play parts in present-day artificial insemination with respect to livestock animals. Extracted semen from desired donor males is emplaced in females artificially. Currently, there are a variety of different conventionally used procedures to make artificial insemination in animals as efficient as possible. The concept of efficiency includes not only minimizing the time involved for each procedure, but also the effectiveness of the procedures to create pregnancies.

Some artificial insemination (sometimes referred to as “AI”) techniques (e.g. dogs) utilize surgery and/or at least anesthesia. This requires a surgical suite for the procedure and a veterinarian. Either procedure usually results in significant trauma to the animal and or its reproductive organs. Trauma is generally not conducive to success in artificial insemination. A non-surgical conventional artificial insemination technique uses what is called a vaginal spirette or catheter that is inserted into the animal's vaginal cavity and directed towards the cervical opening or cranially to its cervix. The spirette has an open channel through its interior and an outer portion with spiral ridges or a foam rubber configuration to gently fit into the cervix. Techniques are used to get the animal to contract the posterior cervix or the muscles of the cervix around the spiral ridges or foam rubber end of the vaginal catheter. A conventional semen tube is then connected to the proximal end of the spirette. With this conventional AI, the semen is drawn through the catheter by the sow's contractions, pulling the semen from the semen tube into the cervix and uterus. The time of the procedure to draw the semen takes approximately 3 to 8 minutes. Optionally a male can be placed in front of the female to start the procedure or during the procedure. The presence of the male is intended to create a physiological response in the female to stimulate the movement of the semen through the cervix to the uterus.

This conventional procedure is not as efficient as desired. It sometimes requires repetition of the procedure because a first time does not always result in all the semen reaching the uterus or a pregnancy. Back flow or leakage of semen draining away from the uterus or cervix has been an industry problem. Approximately one-half of the cost of artificial insemination is the cost of semen. Therefore, loss or use of more semen is a major economic problem. Also, this procedure depends upon the physiological response of the female animal to delivery the semen to the uterus.

Other procedures have been found to have difficulty in navigating, without creating trauma, through the animal's cervical anatomy attempting to gain entrance to the uterus. There is, therefore, room for improvement in the art.

There is a need for improvement in intra-uterine artificial insemination of animals. For example, none of the artificial insemination techniques in swine uses technology traversing the cervical canal and depositing the semen in the uterus with a single one time use catheter.

Also, depositing the semen within the uterus or intra-uterine is desired. Therefore, loss of semen would be prevented, providing the capabilities to decrease the sperm numbers or concentration since the transfer of semen is directly into the uterus. Also, conventional AI procedures depend upon the physiological response of the female animal to delivery the semen to the uterus. A procedure without such dependence would be desirable, thus eliminating the aspect of the loss of semen and time. Other procedures have been found to have difficulty in navigating, with minimum trauma through the animal anatomy to the uterus. There is, therefore, room for improvement in the art.

One attempt to provide such improvement, that has proven successful for sheep, is disclosed at published PCT application W0/97/14365, by one owner of the present application. This publication is incorporated in its entirety by reference herein. The apparatus disclosed in the publication utilizes a lumen that encapsulates an endoscope. A probe end at the end of the lumen has a geometry that is beneficial at navigating the animal's anatomy, particularly the cervix. A relatively small second lumen integrated with or positioned along the first lumen comprises a semen injection canal with an outlet at or near the probe end. The amount of semen used per artificial insemination attempt for these smaller animals is relatively small, and therefore, the semen delivery tube size can be relatively small and still take a reasonable amount of time to inject the semen without damaging the sperm in the semen. If sperm is attempted to be forced through too small of a passageway relative to its volume or is attempted to be moved too fast through a passage, it can experience what is sometimes called turbulence, which can adversely affect, damage or even kill sperm in the semen. The lumen and endoscope are inserted and navigate the animal's tract and cervix, and deposit the semen directly into the uterus. The navigation can be viewed. While this combination works for sheep and small ruminants, there remains room for improvement.

For example, minimal trauma to the animal is generally correlated (directly related to the success of the conception) to the size of the instrument inserted in the animal and the ability to navigate the cervical canal. The distal end is critical in the procedure. The goal is atraumatic navigation and/or entry of the uterine body and traversing the cervix of the animal. Therefore, minimization of size is generally desirable and extremely important to achieve positive results. Such limitation in size limits the diameter or size of the semen injection canal. This limitation in turn limits the throughput of semen. The result is it takes a significant amount of time to inject an appropriate volume of semen (e.g. approximately 90 ml. for sows) for acceptable success rates of pregnancy. Even a savings of a few minutes per animal is very significant.

Each minute spent on one animal takes away the time that could be spent on additional animals. Over the course of hours, this advantage magnifies.

Similar problems exist with a number of other animals. Presently, large-scale swine operations are proliferating. Economic efficiencies are needed for profitability. Improvements in reproduction techniques for such livestock are needed, including artificial insemination procedures.

Still further, the general process for artificial insemination has room for improvement. In some systems each sow is placed in a crate so that it is immobilized. A boar is placed in an adjacent crate, nose-to-nose with the sow. This is done because it is believed to induce a biological response in the sow to increase the chances for successful conventional artificial insemination.

Another technique used in the livestock industry is embryo transfer (sometimes referred to herein as “ET”). Embryos are artificially placed into the uterus of animals. Currently, the technique of placement of embryos in species such as sheep and swine is surgery. This results in the requirement of a surgical suite, veterinarian, and the entire overhead. Present survival rates for emplaced embryos are low in most situations. One cause of low survival rate is due to the trauma experienced by the animal in surgery as well as the lack of expert skill necessary for the placement surgery.

It can therefore be seen that there is room for improvement in the art. It is therefore a principal object of the present invention to provide an apparatus and means for artificial insemination, which improves upon or solves problems and deficiencies in the art. There is particular application for advances in the art relative to placement of semen in sows and gilts, but also in other species.

Further objects, features, and advantages of the present invention include an apparatus and methods for artificial insemination that:

a. Are more efficient

b. Are more economical

c. Can produce less trauma to animals

d. Can be more effective

e. May not require a surgical suite or anesthesia.

Further objects, features, and advantages of the present invention include an apparatus and methods for artificial insemination that:

f. Are more efficient than the two catheter intra-uterine system.

g. Are more economical using less sperm numbers (thus more sow breedings per boar ejaculate) and extender per sow.

h. Can produce less trauma to animals.

i. Can be more effective producing greater conception levels, larger litters and requiring less time per insemination using fresh or frozen semen.

j. Does not require a surgical suite or anesthesia.

k. Requires no electricity.

l. Is a one piece biosafety dispensable intra-uterine catheter.

A further object of an aspect of the invention includes an apparatus and methods for embryo transfer which improve upon or solve deficiencies in the art, and which have the same or similar objects, features, and advantages as previously described regarding artificial insemination.

These and other objects, features and advantages of the present invention will become apparent with reference to the accompanying specification and claims.

### SUMMARY OF THE INVENTION

The present invention includes apparatus and methods for artificial insemination and embryo transfer in livestock. For artificial insemination or “AI”, one apparatus according to the invention includes an elongated inner sheath having a longitudinal channel therethrough and proximal and distal ends. An outer sheath has proximal and distal ends and an internal passageway. The inner sheath slideably fits within the outer sheath. A distal probe end is attached or attachable to the distal end of the inner sheath and is configured to assist navigation of the cervical anatomy of the particular animal involved. Preferably, the probe end has rounded, smooth surfaces, an asymmetrical portion extending outside the perimeter of the inner sheath to which it is attached, and the combination of inner sheath and probe end having a size that allows insertion into the animal and through the cervical anatomy atraumatically, without anesthesia or surgery to the animal. Once the desired position of the distal ends of the probe, inner sheath, and outer sheath inside the animal have been reached, the inner sheath/probe end combination are retracted and removed while leaving the outer sheath in place in the animal. The proximal end of the outer sheath is adapted to receive semen from a semen source. The semen can be moved through the outer sheath to its distal open end, where it can be deposited into the animal.

Optionally, an endoscope can be used during navigation to assist by providing the user with visualization of navigation and position. It can be inserted into the inner sheath. The probe end can be configured to allow the endoscope to have a field of view out of probe end. Once the desired location is reached, the inner sheath/probe end combination and the endoscope can be removed, leaving the outer sheath for delivery of the semen.

An advancement of the present invention is a two-catheter intra-uterine artificial insemination procedure described before. A further advancement is the one time use of a catheter that combines the technology of each to navigate the cervix using a guide probe 66 of the distal tip 62 (exemplary embodiments of which are shown in the appended Figures), however, with an open port 70 to deposit the semen in the uterus. The present invention utilizes the ability to be able to enter the uterine body to deposit the semen without any surgery, restraint or visual requirement. Minimal trauma to the animal is generally correlated to the success of attaining the pregnancy by the size of the instrument inserted in the animal and the ability to navigate the cervical canal. The distal tip 62 with the open port 70 at its distal end is essential to the procedure that allows the atraumatic navigation and entering the uterine body. Therefore, minimization of size is generally desirable and extremely important to the result.

Still further, optionally a vaginal catheter or spirette can be first inserted in the animal. The inner sheath/probe end/outer sheath combination can then be inserted through the passageway through the spirette to navigate the cervical anatomy atraumatically.

Another option is to have a one piece inner sheath/probe end, without an independently moveable outer sheath. An opening can be formed in the distal probe end in communication with a passageway through inner sheath. The single piece device could be used to both navigate the cervical anatomy of the animal, and then deliver semen to the desired location through the internal passageway and out the opening in the probe end. This embodiment is useful when visualization is not needed or desired. Navigation can be achieved by manually manipulating the probe and receiving tactile feedback through the sheath. The second embodiment can be used with a vaginal catheter or spirette. Additionally, the sheath could be strengthened by thickening its walls, fixing another sheath over at least part of its length or otherwise increasing rigidity of the sheath.

A method of AI according to the invention can include navigating through the cervical anatomy of an animal with a probe at the distal end of an inner sheath having the characteristics described above. The probe end/inner sheath combination could be used alone with tactile feedback to the user, or while following the probe end with an outer sheath, with or without visualization feedback by endoscope. Once the probe is in a desired location, semen is delivered to the site. One way is through the inner sheath, where the probe end is given an exit port. Another way is by removing the inner sheath/probe combination, and using the second sheath to deliver semen to the animal for AI.

What is called refractory AI is a method and apparatus for artificially inseminating livestock. For examples, instead of using special AI crates for sows and having a corresponding number of crates for boars to be positioned nose-to-nose to each sow during AI, a relatively large set of sows can be breed quickly, without a boar near the sows during the process. A boar can be walked by the sows to help identify which sows are good candidates for AI, however, the boar is not needed during AI. The sows can be in breeding crates, but do not need to be. AI can then be performed on selected sows. Therefore, only one boar is needed, special crates are not necessary, and it can go much quicker.

A conventional artificial insemination room or area would have several sow crates with a number of boar crates. Significant time, materials, and labor are used to create such a setup, bringing the sows and boars into their respective crates, and performing the procedures. One set of sows (usually one to three per set) is artificially inseminated, they are then removed from the crates, and the next set is then brought into the crates. Under an improvement in the art called herein refractory artificial insemination, sows are also artificially inseminated in their gestation crates one after another without boar contact until, in some cases, after the AI is complete. This could be accomplished while the sows are housed in a large holding area or gestation barn as well. In these barns the boar can be walked in front of the sows after breeding and moved one sow distance at a time to assist in the insemination process, post-insemination.

According to another aspect of the invention, embryo transfer or “ET” can be performed utilizing apparatus and methods that include use of the inner sheath/probe end combination discussed above regarding AI. Embryos are preloaded into a tubular section which is positioned near the probe end. This tubular section can be a part of the inner sheath, or a separate section that is connected to the inner sheath. Navigation, as described with AI, proceeds, using the probe end to atraumatically traverse the cervical anatomy of the animal. One the probe end is in desired position, the embryos can be ejected. One way is to insert a wire or other pushing structure up the inner sheath to push the embryos out an opening on the probe end. Another way is to send pressurized air through the proximal end of the inner sheath to push the embryos out the distal end.

Alternatively, an inner sheath/probe end combination, slideable within an outer sheath, could be used to navigate the cervical anatomy, then removed and a second inner sheath/probe end combination with preloaded embryos inserted through the outer sheath. Additional navigation is therefore possible because of the probe end, but when in position, the embryos are then ejected through an opening in the probe end of the second inner sheath/probe end combination.

### BRIEF DESCRIPTION OF THE DRAWINGS

FIG. 1 is a perspective view of an embodiment of the present invention with portions cut away and diagrammatically illustrating attachment to an endoscope.

FIG. 2 is an enlarged sectional view taken along the line indicated as “FIG. 2” in FIG. 1.

FIG. 3 is an enlarged sectional view taken along the line indicated as “FIG. 3” in FIG. 1.

FIG. 4 is a plan view of the inner sheath; probe end, outer sheath, rotational connector and endoscope of FIG. 1.

FIG. 5 is the same as FIG. 4 without the endoscope but includes a vaginal foam catheter over the outer sheath.

FIG. 6 is an enlarged sectional view of the distal end of instrument 10 in FIG. 1.

FIG. 7 is an enlarged partial sectional view of the proximal end of the instrument of FIG. 1.

FIGS. 8-18 are various views of the endoscope, connector and rotational connections of FIG. 7.

FIG. 19 is an enlarged sectional view of the probe end of FIG. 1.

FIG. 20 are enlarged side elevation views of the rotational connector of FIGS. 4 and 5.

FIG. 21 is a diagrammatic view of the probe end of FIG. 1.

FIG. 22A is an enlarged longitudinal sectional view of the portion of distal tip of FIG. 1.

FIGS. 22 B-D are side, bottom, and end views of a portion of the distal tip of FIG. 1.

FIG. 23 is a diagrammatic view of endoscope equipment of the type that could be used with the embodiments of the present invention.

FIG. 24 is an exploded view of the components of FIG. 1 and option additional components useful with the embodiment of FIG. 1.

FIG. 25 is a side view of the inner and outer sheaths with a spirette vaginal catheter.

FIG. 26 is a side view of the outer sheath and the foam vaginal catheter.

FIG. 27 is an enlarged sectional view of the distal second inner sheath and distal tip.

FIG. 28 is a diagrammatic exploded view of endoscopic equipment and sheaths of the type that could be used with the embodiments of the present invention embryo transfer.

FIG. 29 is a side view of the complete second inner sheath and 3^{rd} outer sheath and innermost embryo sheath used for embryo transfer.

FIG. 30 is similar to FIG. 29 but shows the second inner sheath and 3^{rd} outer sheath and innermost embryo sheath of FIG. 29 inserted in a vaginal spirette.

FIG. 31 is a depiction of an embodiment of the two-sheath artificial insemination invention within the cervix and uterus of an animal.

FIG. 32A is an enlarged longitudinal sectional view of the distal portion of FIGS. 29-30.

FIGS. 32 B-D are side, bottom and end views of a portion of the distal tip in FIGS. 29-30.

FIGS. 33A-C are a top diagrammatical plan and view of a prior art of an artificial insemination according to the embodiment of the present invention.

FIG. 34 is a perspective view of an embodiment of the present invention with portions cut away and diagrammatically illustrating it and the vaginal catheter 42.

FIGS. 35 is the same as FIG. 34 but diagrammatically illustrating only the catheter 101.

FIG. 36 is an enlarged plan view of the sheath and distal tip end 102, the same as FIG. 34 but diagrammatically illustrating only catheter 101 in FIG. 34.

FIG. 37 is a diagrammatic view of the embryo transfer sheath system placing embryos in the uterine horn in relationship to the other organs.

FIG. 38 A is an enlarged longitudinal sectional view of the portion of distal tip 62.

FIGS. 38 B-D are side, bottom, and end views of a portion of distal tip 62.

FIG. 39 is a diagrammatic view of the catheter 101 within the cervical canal of a sow.

### DETAILED DESCRIPTION OF THE PREFERRED EMBODIMENTS

Examples of forms the invention can take will now be described with reference to the accompanying Figures. Reference numbers and letters will be used in the drawings to indicate certain parts and locations in the drawings. The same reference numbers and letters will be used to indicate the same parts and locations throughout the drawings unless otherwise indicated.

The drawings are not necessarily precisely to scale. Dimensions have been added to some of the drawings for the example embodiments discussed. Dimensions could vary consistent with the invention.

A. Artificial Insemination

1. First Embodiment

The first embodiment will be described with regard to artificial insemination of sows or gilts.

FIG. 1 illustrates instrument 10 configured to begin artificial insemination. An inner sheath 12 (e.g. approx. 24 ½″ long and 0.086″ or 2.18 mm o.d. clear plastic) has a distal end 14 to which is glued or sonified a distal tip 16 of micro-molded clear plastic. Distal tip 16 is attached around the outside of distal end 14 by EPO TEK 302-type epoxy glue if glued. Proximal end 18 of inner sheath 12 includes a funnel-shaped end piece 22.

Endoscope fiber optics 20 are insertable through end piece 22 of the inner sheath 12 up to the distal tip 16 and include a camera 32 and camera fiberoptics 24 and light fiberoptics 26 connected to endoscope 29 (connected to fiberoptic light cable by a light port connection 31) with a fiberoptic light cable 30 connected to the light source 34.

Inner sheath 12 is sealed and fluid impermeable, as is distal tip 16. Endoscope fiber optics 20 is slideably insertable and removable so that they can be reused in multiple inner sheaths 12.

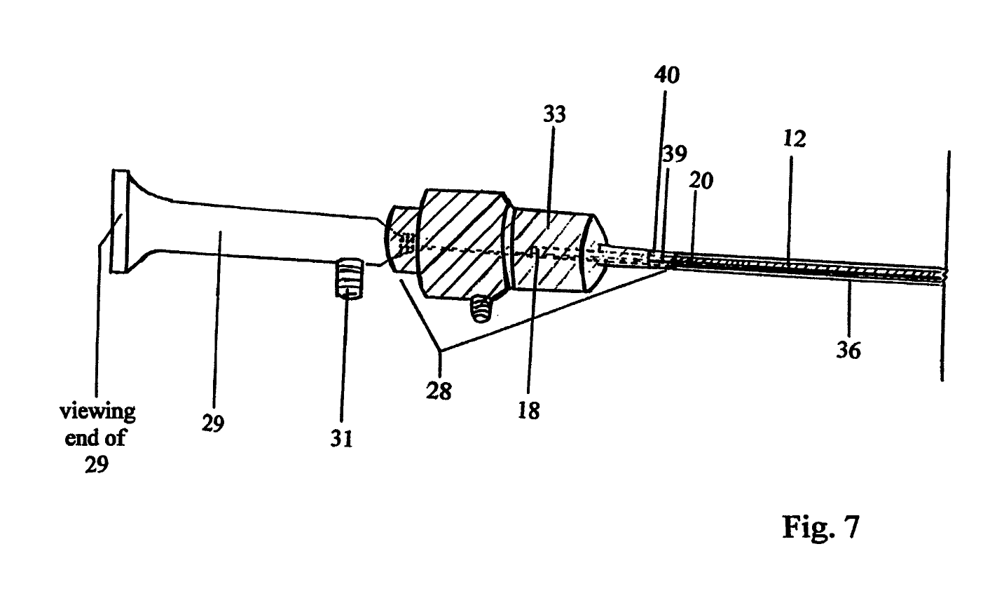

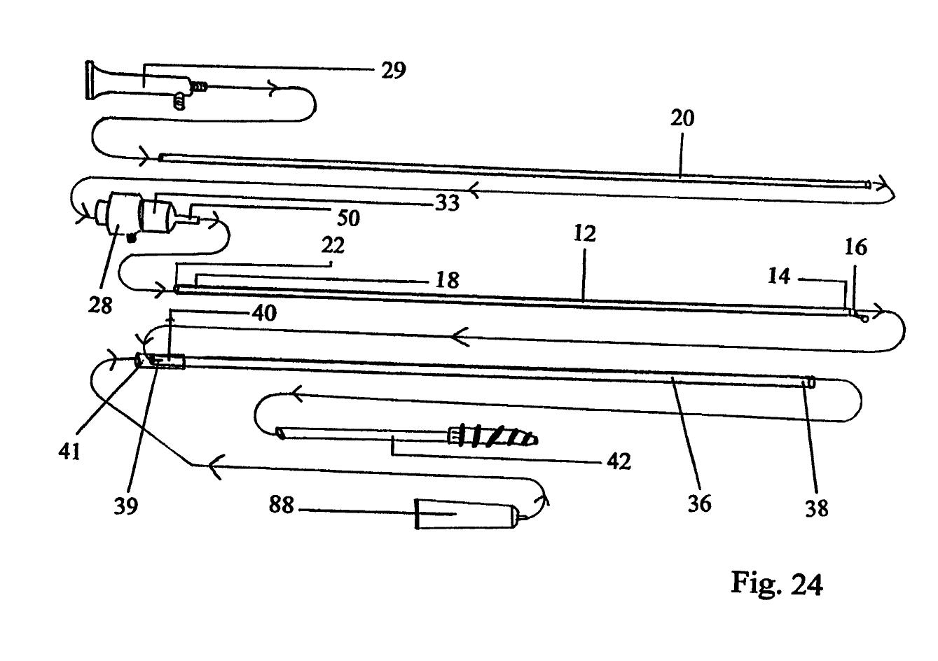

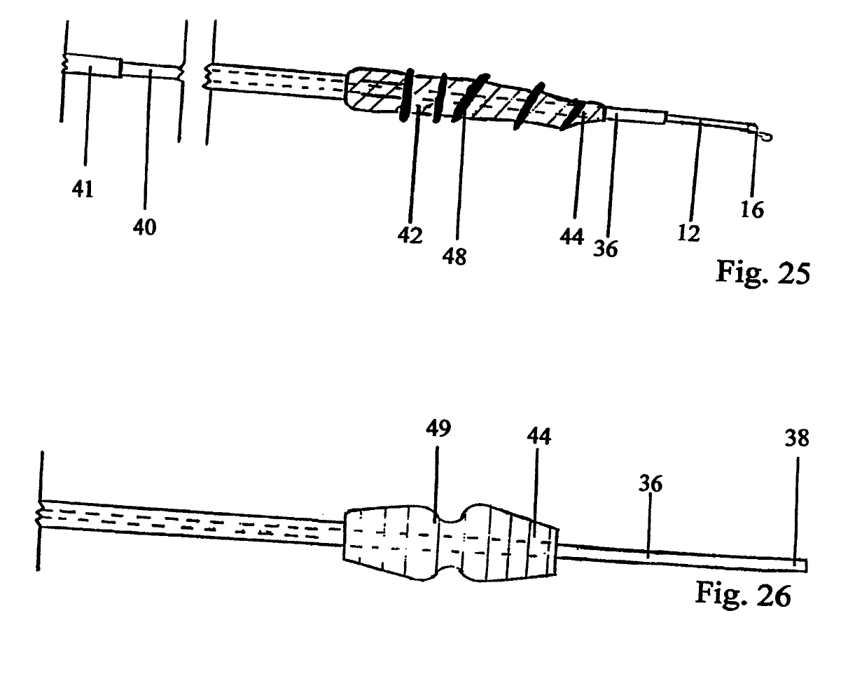

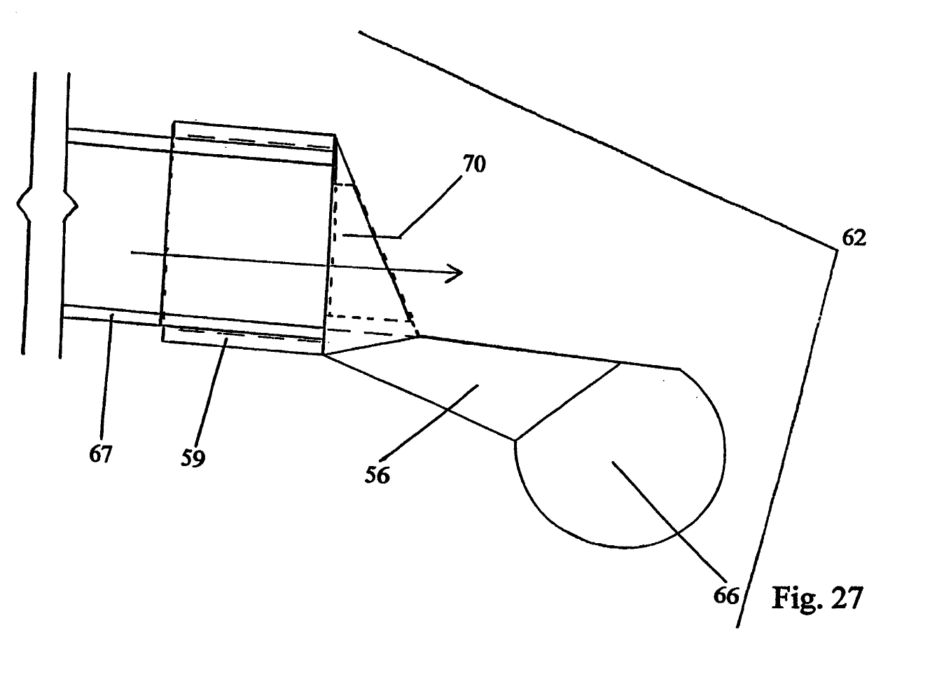

Inner sheath 12 is slideably insertable through an outer sheath 36 (e.g. approx. 23½″ long, 0.196″ or 4.98 mm o.d. clear plastic). Distal end 38 of outer sheath 36 is open, as is proximal end 40. Proximal end 40 has the longitudinal ¼″ slit 39 (FIGS. 7 and 24) and force fits upon fitting 50 on a connector 28.

Outer sheath 36 slideably fits within the vaginal spirette catheter 42 or foam catheter 49 that has been cut to approximately 12-16″ in length after being placed in the animal having distal ends 44 and proximal ends 46 which are open and a plastic over fitting having spiral exterior 48 on the spirette catheter 42 or foam exterior on catheter 49.

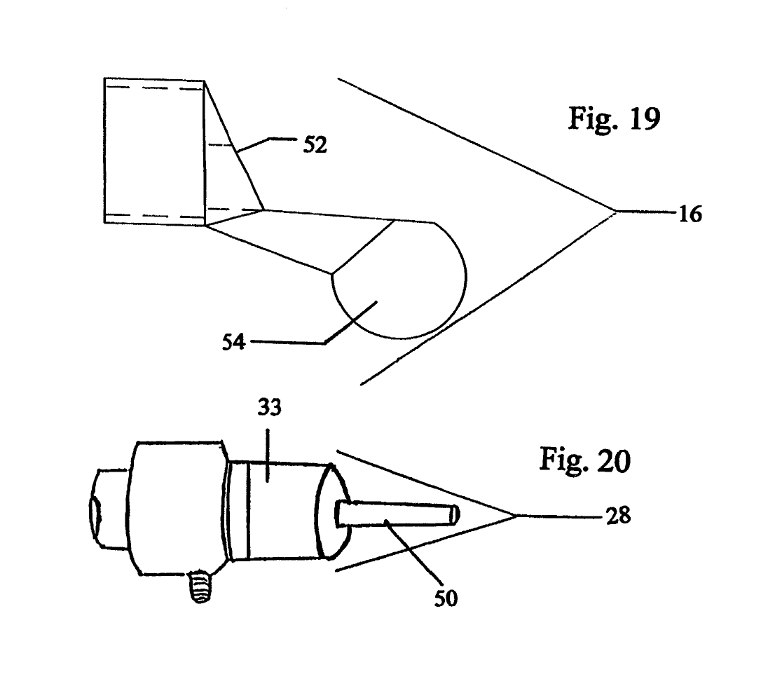

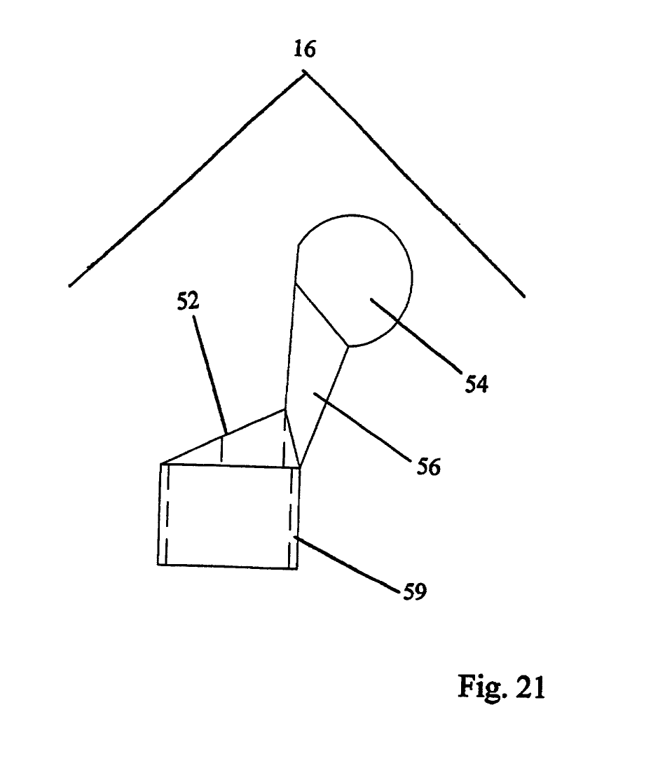

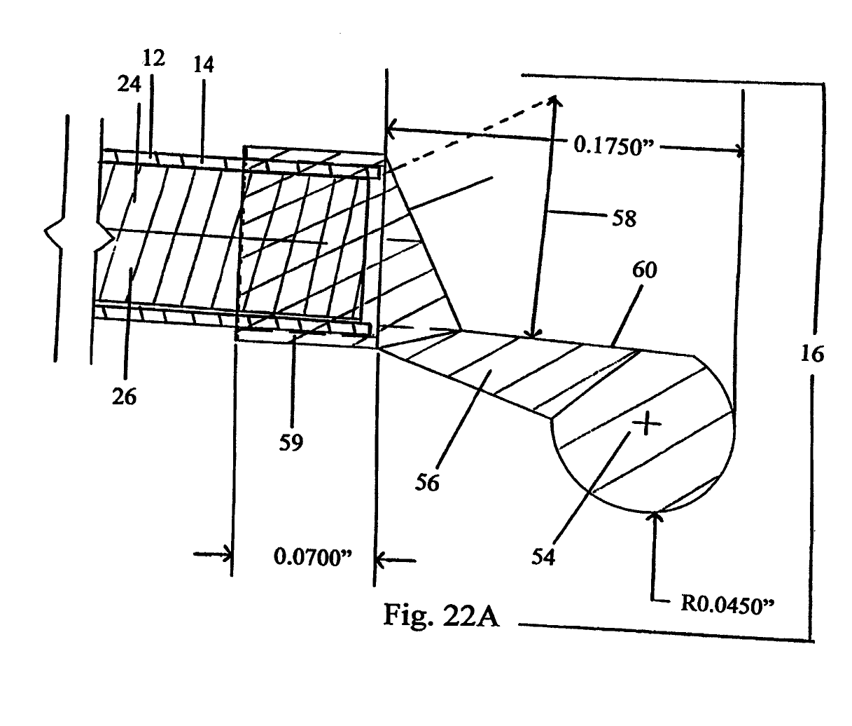

The arrangement of FIG. 1 includes these characteristics. The distal end of endoscope 29 fiber optics 20 are slideable up to distal tip 16 of inner sheath 12 and provide a depth of view of approximately 1-3 mm looking forward of distal tip 16 through a clear plastic window 52 in distal tip 16 (See FIG. 19, 22A-D).

Inner sheath 12 is longitudinally slideable relative to outer sheath 36. Once installed in outer sheath 36 and with coupler 28, both outer and inner sheaths rotate together because of the closeness of inside diameter of the outer sheath and outside diameter of the inner sheath. Outer sheath 36 has been sized to closely conform with the outer diameter of inner sheath 12 to minimize the outer diameter of outer sheath 36, yet allow the slideable movement of inner sheath 12 relative to outer sheath 36. Because guide probe tip 54 of distal tip 16) extends outside the outside diameter of inner sheath 12, a small longitudinal slit 39 (FIGS. 7 and 24) [e.g. approximately ¼″]) is made to proximal end 40 of outer sheath 36 to facilitate entry of distal tip 16 into outer sheath 36 while minimizing risk of breakage of guide probe 54. The very outer surfaces of outer sheath distal end 38 are polished to be atraumatic for a presentation of that end when being inserted.

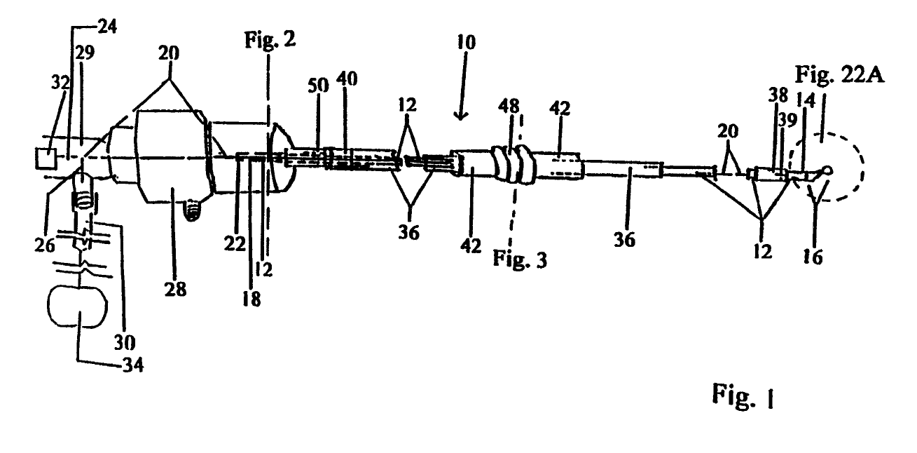

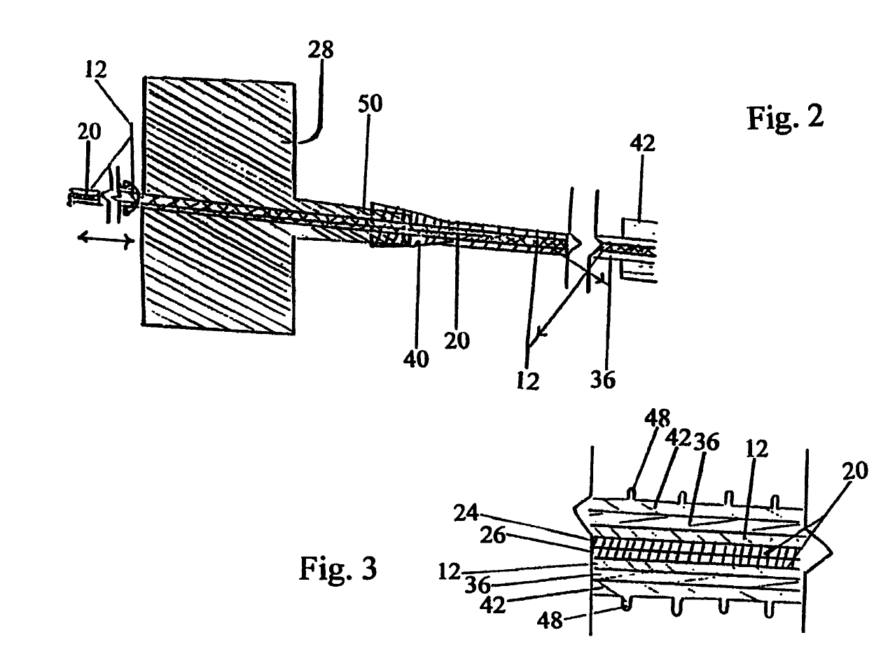

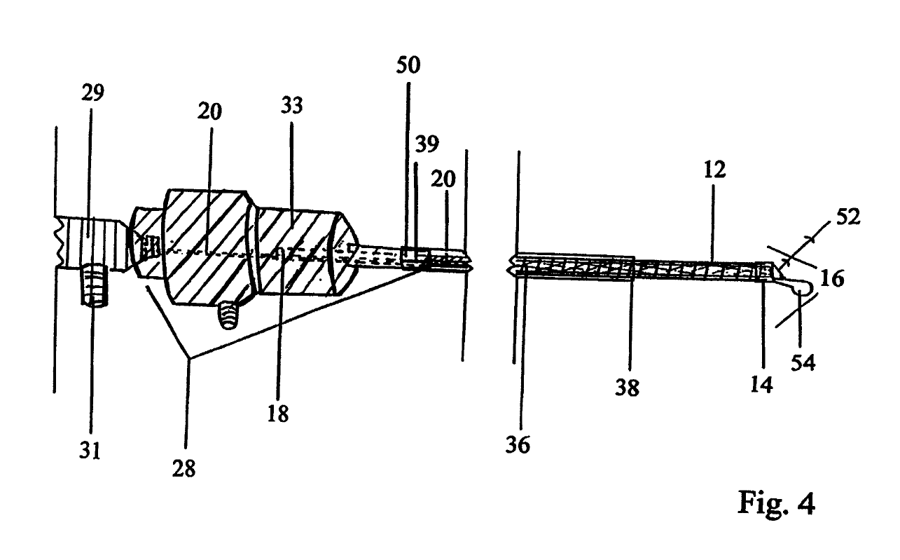

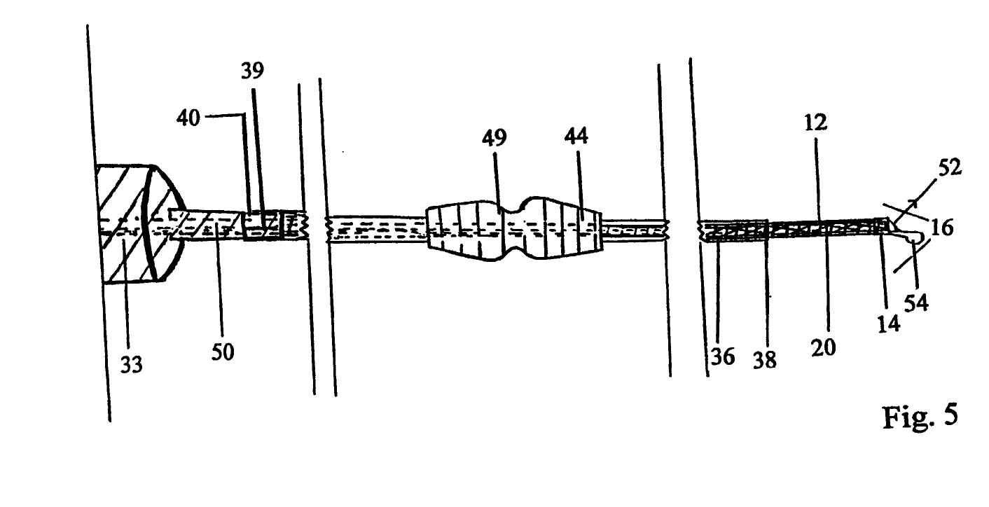

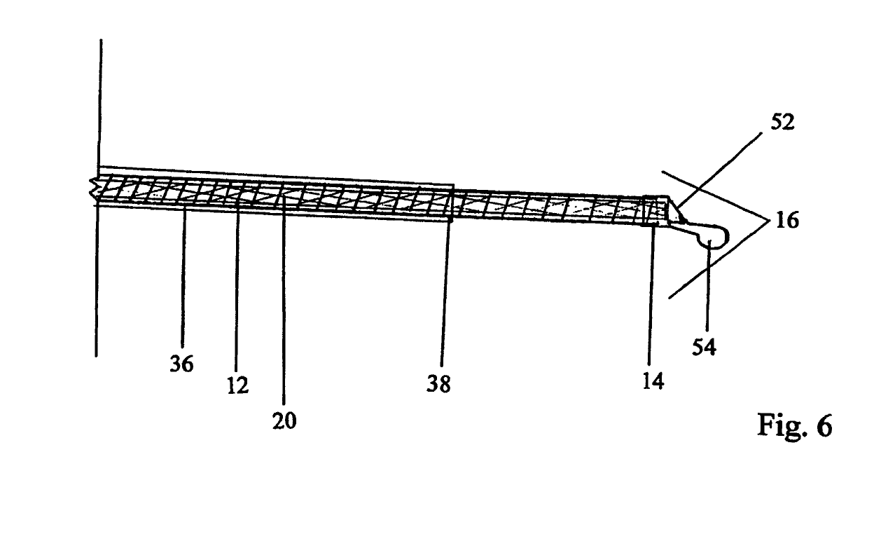

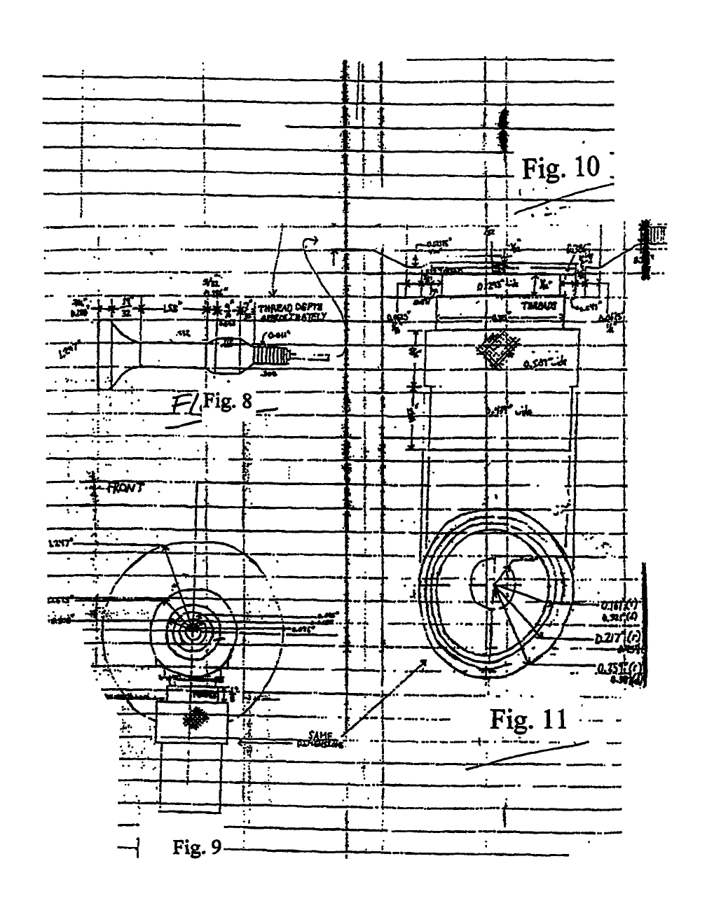

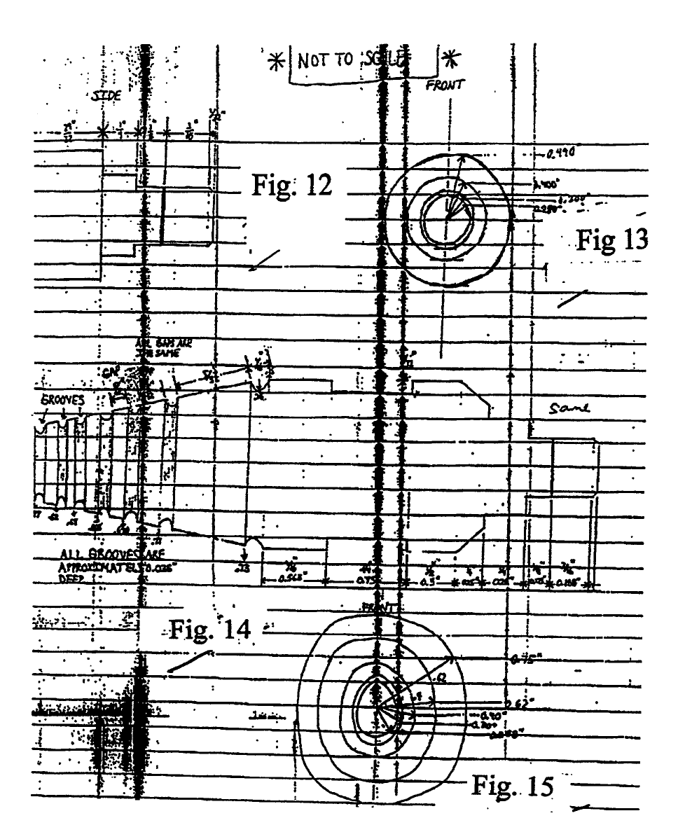

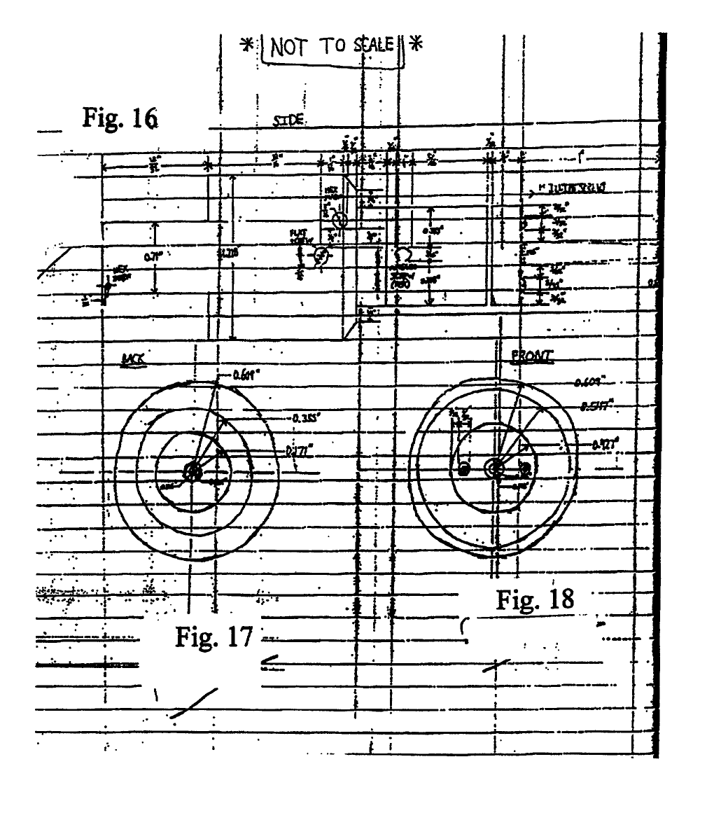

Importantly, inner sheath 12 can be manually manipulated during the insertion process to rotate it and longitudinally move it inwardly and outwardly to facilitate navigation of the anatomy of the animal, including the cervical canal. The guide tip 54 geometry provides rounded surfaces, yet the asymmetrical offset from the diameter of inner sheath 12 to essentially be the atraumatic finger that can probe and gently move tissue as well as assist in atraumatic navigation. The size of distal tip 16 and inner sheath 12, as well as outer sheath 36, are approximately the size of and not much larger than the smallest diameter section of the cervical canal. Outer sheath 36 strengthens and supports inner sheath 12. Its outside diameter is also relatively small to minimize trauma and ease navigation without anesthesia to the animal. Outer sheath 36 connection to fitting 50 on connector 28 makes it easier for the inseminator to manipulate the entire instrument. FIGS. 2-20 show features of instrument 10 in additional detail.

It is to be understood that it is many times desirable to minimize the size of the probe end and sheaths that traverse the cervix, so as to minimize any trauma to the cervix or other parts of the reproductive tract. Yet, it is many times desirable to have sufficient size to move or manipulate parts in the cervical canal. Further, it is many times desirable to maximize the size of the delivery passageway for semen through the sheath and probe end, so as to minimize turbulence or other damage to the sperm in the semen. These factors impact the design of the embodiments described herein. For the specific animal involved, here swine, the outside diameter of the sheath is on the order of 3.7 mm, in one embodiment. The probe end is smaller than that in diameter, but extends outside the perimeter dimensions of the sheath 12. This structure allows the distal end of sheath 12 and the probe end to form sort of a wedge that props parts of the cervical canal open as they move through, while at the same time being small enough so that they are atraumatic to the cervical canal. The finger-like probe end also allows lifting or moving or pushing of tissue, if needed, to help navigation, again without trauma. At the same time, the inside diameter of sheath 12 is sufficiently sized that the substantial volume of semen usually injected into sows during AI can be moved at a reasonable rate without turbulence, for quick and effective AI. It is believed if the smallest diameter of the path through the animal's cervical canal is one-half or less than the outside diameter of the AI instrument traversing it, that it can cause significant damage to the cervical structure or at least trauma to reduce the probability of success of the AI. Thus, generally for sows, the outside diameter of sheath 12 is around 3.7 mm, whereas the smallest diameter across the cervical canal is about 1 mm to 3 mm. Therefore, the outside dimensions of the instrument 10 should probably not exceed 2 to 3 times the smallest diameter across the cervical canal.

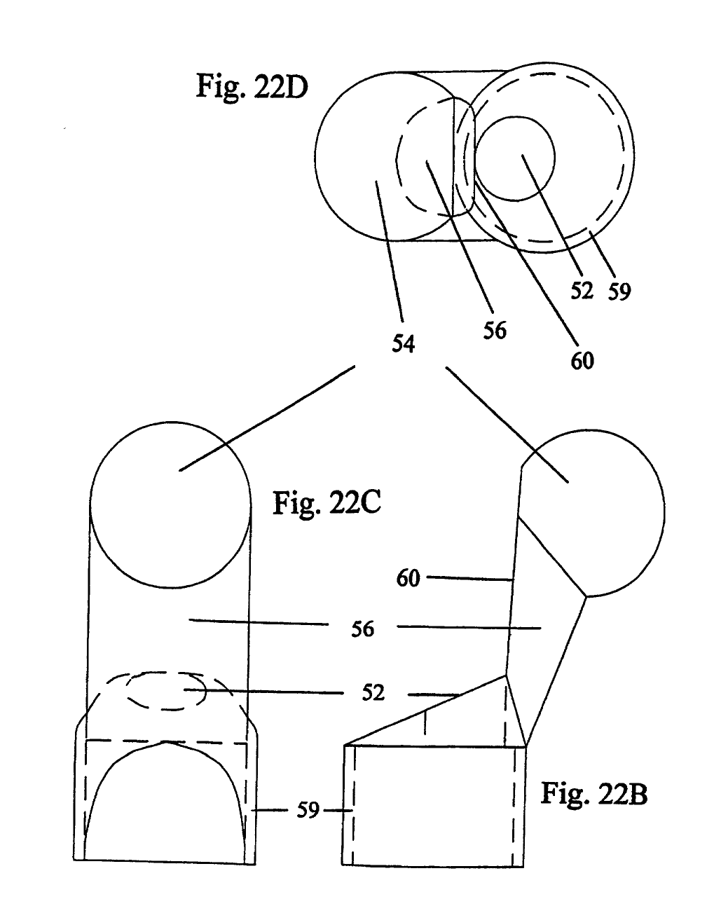

FIGS. 22A-D illustrates in more detail the specific shape and geometry of distal tip 16. Guide probe 54 has dimensions shown in FIG. 22A. A tapered section 56, with rounded surfaces (See FIGS. 22B-D) connects the tip guide probe 54 to a tubular cap 59 glueable or sonified to the distal end of inner sheath 12. Field of view 58 of camera optic 24 for the endoscope is illustrated in FIG. 22A. A flattened portion 60 of guide probe 54 maximizes field of view 58. It is to be understood that reflections off flattened portions 60 from light fibers 26 show up in the image on the endoscope visual display as a sort of corona or halo, or otherwise visually perceivable shading or variation in gray scale. Therefore, without actually having a portion of guide probe 54 obstruct field of view 58; the operator can perceive the relative position of guide probe 54 in the field of view 58. This assists in navigation because it provides the operator with information regarding the relative position of guide probe 54.

It is again mentioned that all surfaces of distal tip 16 are as rounded and polished as possible for minimization of trauma to the animal.

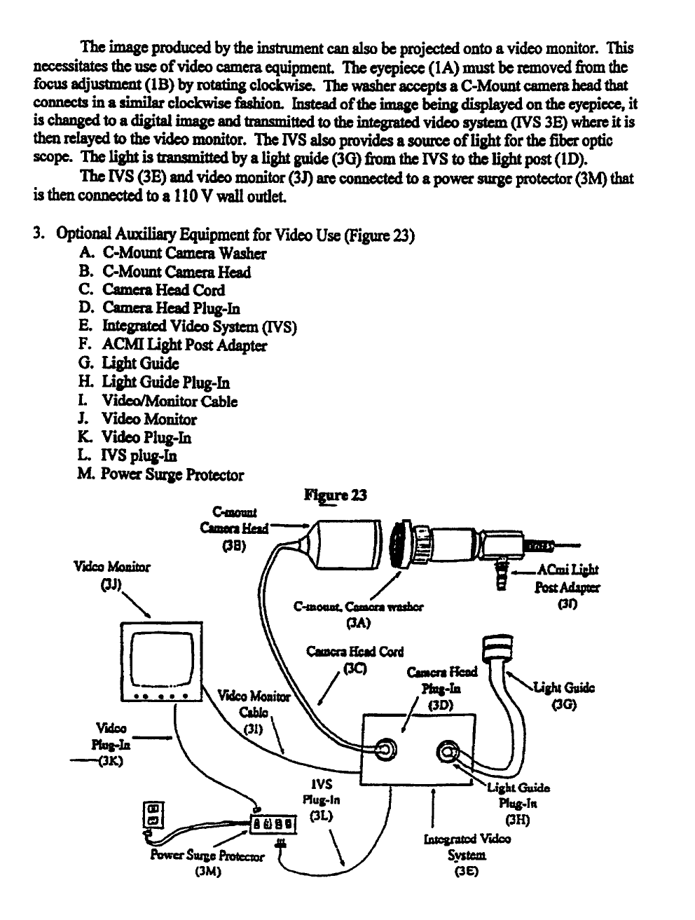

FIG. 23 illustrates the components that are used for the endoscope 29. Endoscopes 29 and 20 are conventional and available from a variety of vendors.

FIG. 24 illustrates endoscope 20 (distal length), inner sheath 12, outer sheath 36, spirette 42, and connector 28 as previous described in exploded form.

The method of artificial insemination is now described. Instrument 10 is designed to pass through (traverse) the cervical canal of a sow or gilt to deposit the spermatozoa directly into the sow's uterus. The animal is confined to a breeding crate, however she is not restrained or anesthetized. Instrument 10 is assembled as shown in FIGS. 1 and 7, distal length of the endoscope 20 inserted into inner sheath 12 and the endoscope 29 connected to the endoscopic light source 34 by the fiberoptic light cable 30. Coupler 28 has a cylindrical rotating portion 33 that rotates allowing the outer and inner sheaths to rotate with it and is threaded to endoscope's proximal end 29. Instrument 10 is inserted into the vulva of a sow or gilt by first placing the conventional vaginal spirette 42 in the sow cranially to the cervix as a guide to the cervix (approx. 12″ to 14″ into a sow). Catheter 42 is available from a number of manufacturers or distributors. Distal end 44 has an outside diameter that can slideably receive outer sheath 36.

As with conventional AI, the spiral ribbings on part 48 of spirette 42 would be firmly held into place when the animal contracts onto the ribbings and then instrument 10 traverses the cervix. During such navigation, the operator utilizes the image produced by the endoscope to view the cervix at the point of distal tip 16. The operator uses a side-to-side rotating or a counter clockwise motion to assist traversing the cervical canal containing the cervical folds (interdigitating prominences [“IP”]). The guide probe 54 of distal tip 16 navigates through the cervix's interdigitating prominences (approximately two to three inches in length in sows) using the rotating or counter clockwise motion to the desired location in the uterine body. Once the location is achieved, the endoscope 29 and 20 with connector 28 are removed. Then the inner sheath 12 with distal tip 16 is completely removed by pulling it out through the proximal end of outer sheath 36 so only the outer sheath 36 is the remaining part of instrument 10. A conventional semen tube 88 in FIG. 24 can be attached to the proximal end of outer sheath 40 by semen tube connector 41 and semen deposited within the uterus of the animal.

Outer sheath 36, spirette 42, and semen tube 88 would be withdrawn from the animal and the artificial insemination procedure is complete.

It is also to be understood that the present invention gives tactile feed back to the user to assist in knowing where the distal end of the instrument is relative to the reproductive tract. The user can use the probe end like a finger to feel the way through the cervical canal. In some cases, the probe end literally audibly gives a popping noise as it passes cervical structure; which is also good feed back to the user.

This intra-uterine artificial insemination process places swine spermatozoa directly into the uterus to increase conception rates and litter sizes. The small diameter inner sheath 12 and distal tip 16 minimizes trauma during navigation to the appropriate uterine spot, but provides vision and the manipulation tools to accurately and quickly get to the correct area.

Withdrawal of inner sheath 12 leaving outer sheath 36, gives a relatively large diameter conduit to inject the semen that reduces time. Depositing semen directly in the uterus and the relatively small diameter of outer sheath 36 minimizes trauma and back flow of semen during injection, as compared to larger artificial insemination instruments that only enter the first part of the cervix. Entry into only the beginning of the cervix can lose up to 50% of semen deposited, which results in that amount not being “absorbed” and wasted or lost as in the conventional AI process. Semen represents on the order of one-half the cost of artificial insemination. Therefore, this lost semen is expensive and reduces the total effectiveness of the conventional artificial insemination procedure. Using the intrauterine procedure with the invention described, little or no semen is lost due to back flow because it is deposited directly into the uterus. Thus, decreased sperm total numbers or concentration can be utilized because of direct intrauterine deposition.

Therefore, there is increased reproductive performance by the invention that uses semen, including frozen semen, creating increased pregnancies with reduced spermatozoa numbers per dose, and less total volume and extender needed, less time in the insemination barn, with greater number of baby pigs born.

Conventional artificial insemination methods rely on the animal to move the semen through the cervix after the procedure is completed.. This can take several minutes, if not more time, is not as efficient as the invention and sometimes fails and requires a re-try.

2. Second Embodiment

This embodiment will be described with regard to intra-uterine artificial insemination of sows or gilts.

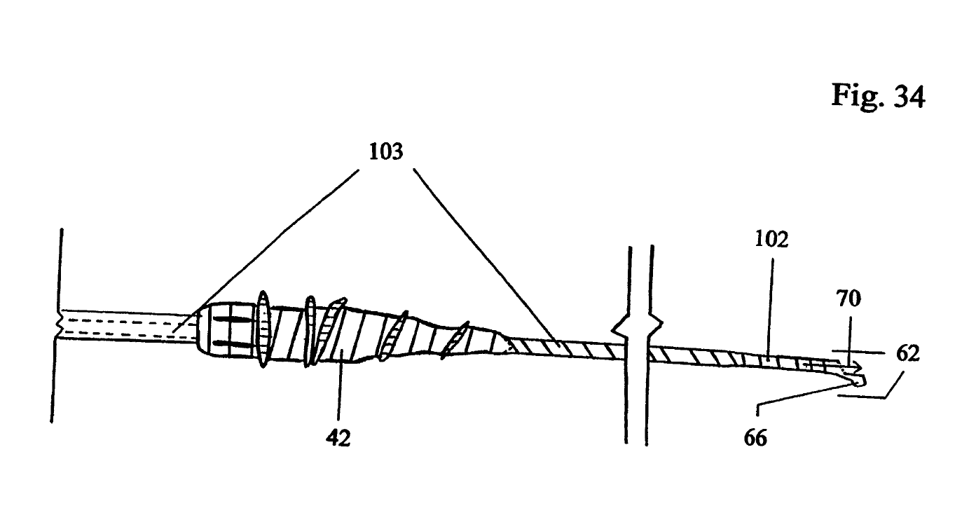

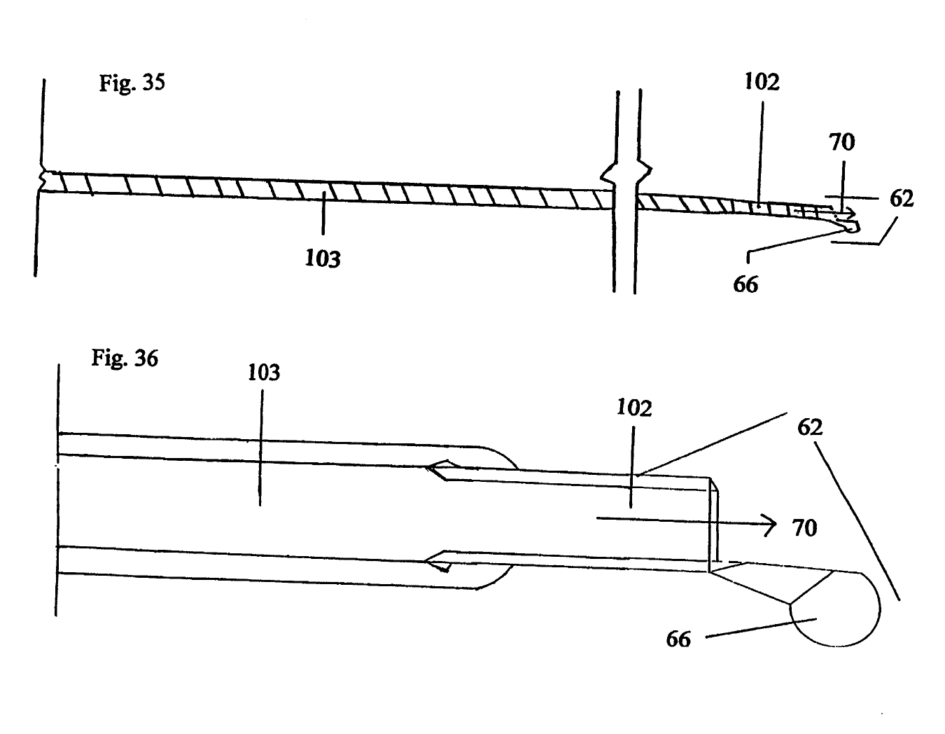

FIG. 34 illustrates the one-piece intra-uterine breeding catheter 101 to achieve the artificial insemination. The intra-uterine breeding catheter 101 is approximately 30 inches in total length. The proximal end 103 is approximately 3.75 mm or 0.15″ o.d. in diameter for 29 inches in length and has an i.d. (internal diameter) of approximately 2.70 mm or 0.10″. The distal end 102 of catheter 101 is 15/16-1 inch in length and is narrowed down to approximately 2.80 mm or 0.086″ o.d. (which is slightly larger than the o.d. of the inner sheath 62 of the two sheath intra-uterine system described in the first embodiment of AI, above), and an i.d. of approximately 1.67 mm or 0.066″. It has the same dimensional distal end tip 62, and guide probe 66 as in the two-sheath system that has the same approximate geometry. However, the distal tip 62 has an exiting port 70 for the semen to be injected into the uterus.

The intrauterine catheter 101 slideably fits longitudinally within the vaginal spirette catheter 42 or foam catheter 49 that has been cut to approximately 12-16″ in length after being placed in the animal having distal ends 44 and proximal ends 46 which are open and a plastic over fitting having spiral exterior 48 on the spirette catheter 42 or foam exterior on catheter 49.

The arrangement of FIG. 34 includes these characteristics.

The very outer surface of distal tip 62 is polished for a presentation of that end to be inserted atraumatically through the cervical interdigitating prominences (cervical rings) and into the uterus.

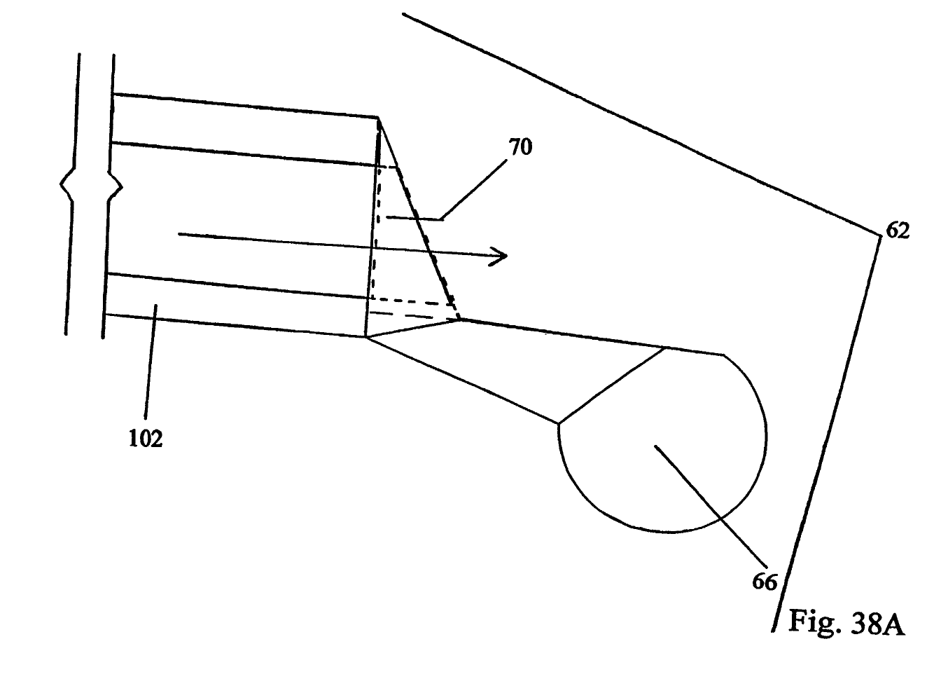

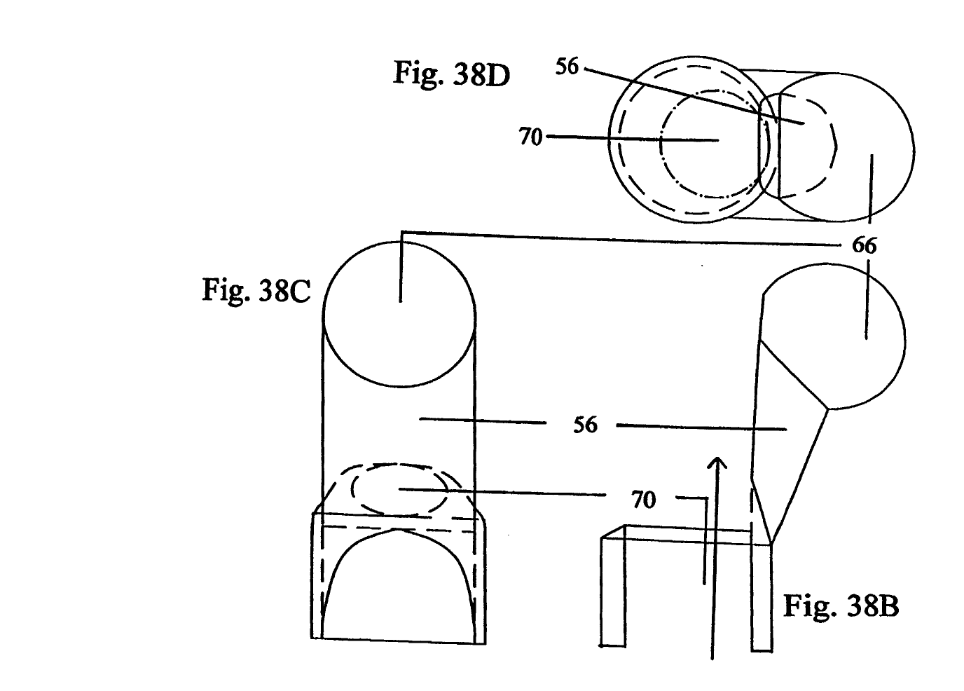

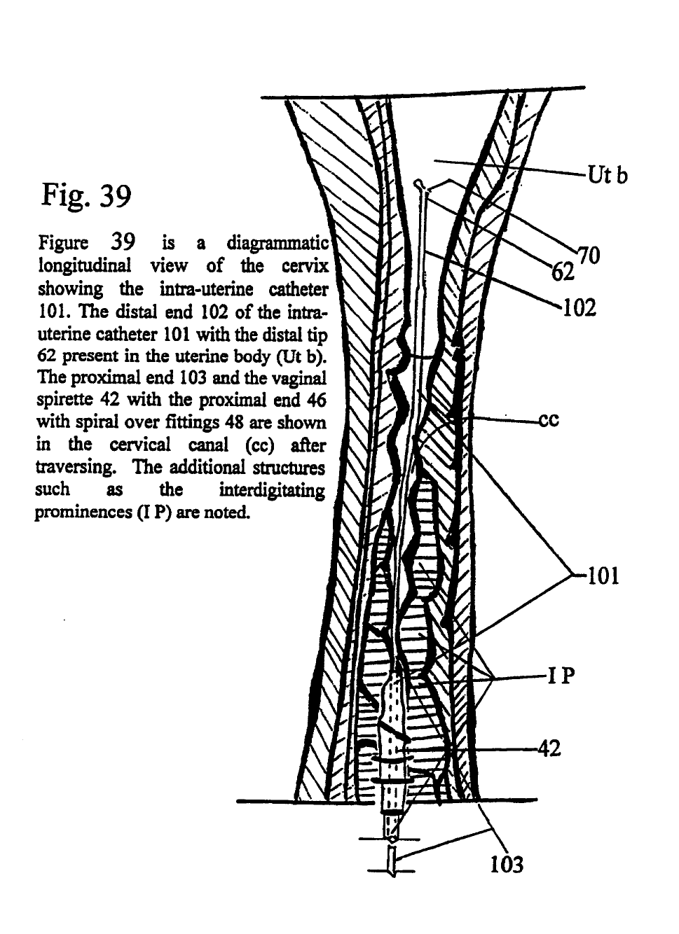

Importantly, the intra-uterine catheter 101 can be manually manipulated during the insertion process by rotating it and longitudinally moving it inwardly and outwardly to facilitate navigation of the anatomy of the animal, including the cervical canal. The guide probe 66 geometry provides rounded surfaces, yet the asymmetrical offset from the diameter of the length of catheter 101 is to essentially be an atraumatic finger that can probe and gently move tissue aside as well as assist in atraumatic navigation. The size of the intra-uterine catheter 101 and distal tip 62 are approximately the size of and not much larger than the diameter of the cervical canal. Its outside diameter is also relatively small to minimize trauma and ease navigation without anesthesia to the animal. FIGS. 38 A-D illustrates in more detail the specific shape and geometry of distal tip 62 that is the same as for the two-sheath embodiment previously mentioned. Guide probe 66 has dimensions shown in FIG. 38 A. A tapered section 56, with rounded surfaces (FIG. 38 B) connects guide probe 66 to the distal end in a similar fashion as in other guide probe dimensions with attachments 59 that is all part of the molded distal end 102 of the intra-uterine catheter 101. It is again mentioned that all surfaces of distal tip 62 are as rounded and polished as possible for minimization of trauma to the animal.

The method of artificial insemination using the intra-uterine catheter 101 is now described. The instrument intra-uterine catheter 101 is designed for the distal end 102 to facilitate passage through the cervical canal of a sow or gilt and deposit spermatozoa directly into an animal's uterus without any visualization aid, performing it only by feel or tactile sense. The animal, determined to be in proper estrus, is confined to a breeding crate to be artificially inseminated, however she is in a free standing position without any restraint or anesthesia. A vaginal catheter 42 (approximately 23 ½ inches in length) is inserted into the vulva of a sow or gilt and used as a guide to approach the cervix (approximately 12-14 inches from the sow's vulva). Vaginal spirette 42 or foam catheter 49 is available from a number of manufacturers or distributors. The proximal end of 42 or 49 have an outside diameter that can longitudinally and slideably receive intra-uterine catheter 101.

Once insertion has gone to the point of conclusion, rotation of the vaginal catheter in the cervix is preferred than pushing it too far into the cervix. The spiral ribbings 48 of catheter 42 will be securely tightened into place when the animal contracts onto it. When such contractions firmly hold the vaginal catheter 42 in place, the intra-uterine catheter 101 is inserted into the proximal end of spirette 44 until it reaches the animal's cervix. Then, intra-uterine catheter 101 is used to traverse or navigate the cervix in which the operator utilizes the tactile sense of the point of tip 62 and distal end 102 of the catheter. A side-to-side rotational motion or counter clockwise movement is used to assist in traversing the cervical canal (containing the interdigitating prominences) and enter the uterus. The guide probe 66 navigates (tactilely sensing the “pop” or guidance when passing each of the interdigitating prominences) through the cervix (approximately two to three inches in length in sows). Once traversing of the cervix is completed there is a freefalling movement without any resistance that signals to the inseminator the entrance and the desired location in the uterus. Once the location is achieved, a conventional semen tube 88 can be inserted or attached to the proximal end of the intra-uterine catheter 101 and semen is deposited through exit port opening 70 as shown in FIG. 38 into the uterus of the animal.

Once the semen is placed in the uterus the intra-uterine catheter 101, vaginal catheter 42, and semen tube 88 are withdrawn from the animal and the artificial insemination procedure is completed.

This process places spermatozoa directly into the uterus that decreases the amount of sperm concentration or sperm numbers needed, as well as increases conception rates, litter size, decreases the time of insemination and eliminates back flow and loss of semen. The small diameter of intra-uterine catheter 101 and distal tip 62 minimizes trauma during navigation to properly and quickly get to the desired location.

Depositing semen directly in the uterus and the relatively small diameter of the intra-uterine catheter 101 minimizes trauma and back flow of semen during deposition that larger artificial insemination instruments can only enter the beginning of the cervix and must have the assistance of the sow to get the semen to the uterus. Such semen losses can account for up to 50%, which results in that amount not being “absorbed” when deposited. Semen represents on the order of one-half the cost of artificial insemination. Therefore, this lost is expensive and reduces the potential effectiveness of the artificial insemination. This invention deposits the semen directly into the uterus thus eliminating any or all semen backflow and loss. Since minimal semen is lost, sperm numbers (concentration) and extender volume can be decreased because of the direct intra-uterine deposition. It can prevent the loss of each.

Conventional methods rely on the animal to move the semen through the cervix. This can take several minutes, if not more time, and sometimes fails and requires a re-try.

Therefore, there is increased reproductive performance placing the semen in the proper location of the uterus using a one piece intra-uterine depositing fresh and extended semen or frozen semen that creates increased pregnancies with reduced spermatozoa numbers per dose, less extender volume needed, less time per insemination, with a greater number of baby pigs born per sow insemination.

B. EMBRYO TRANSFER

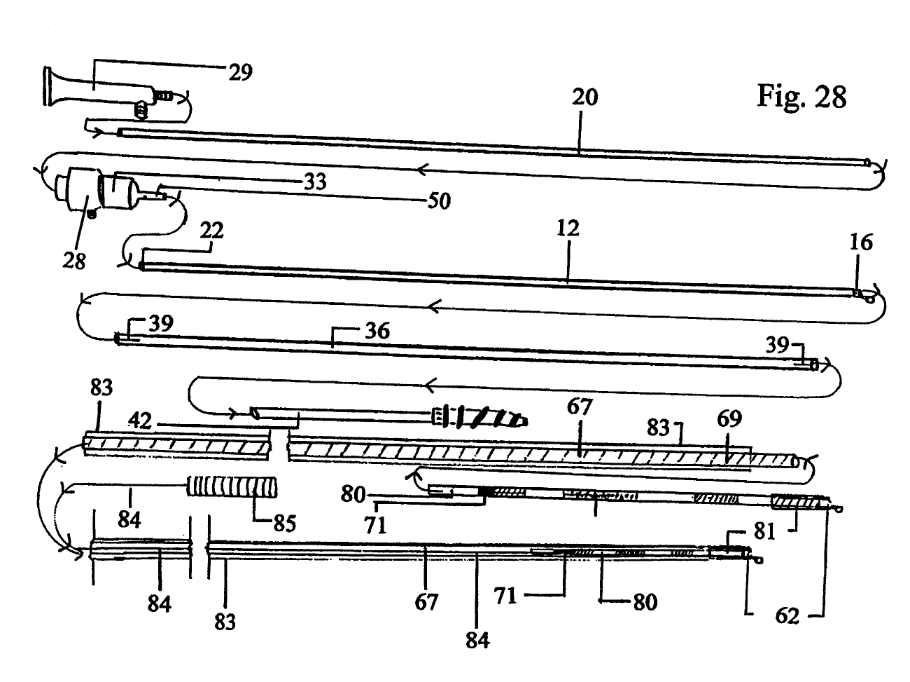

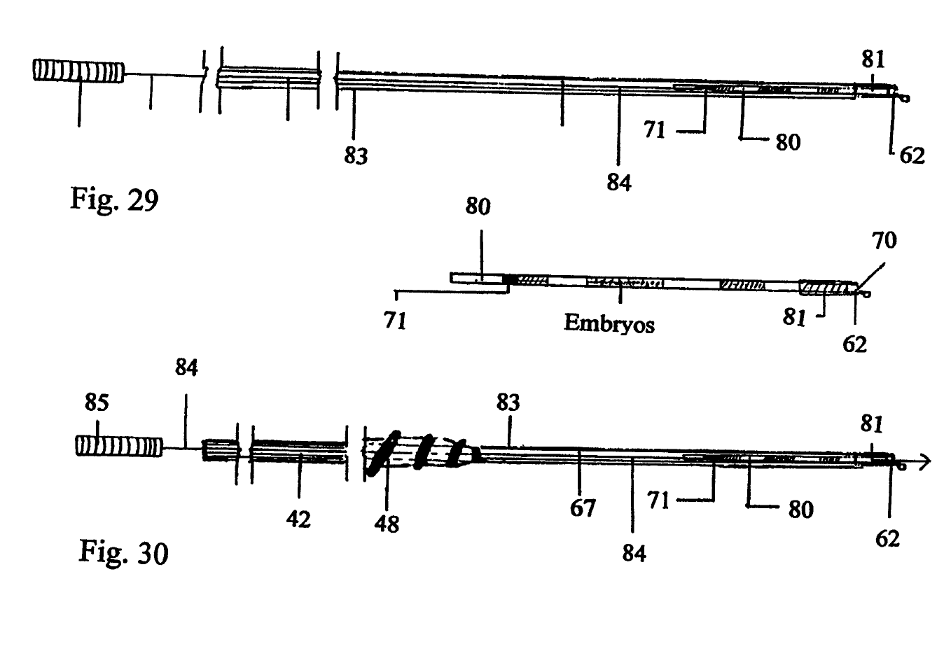

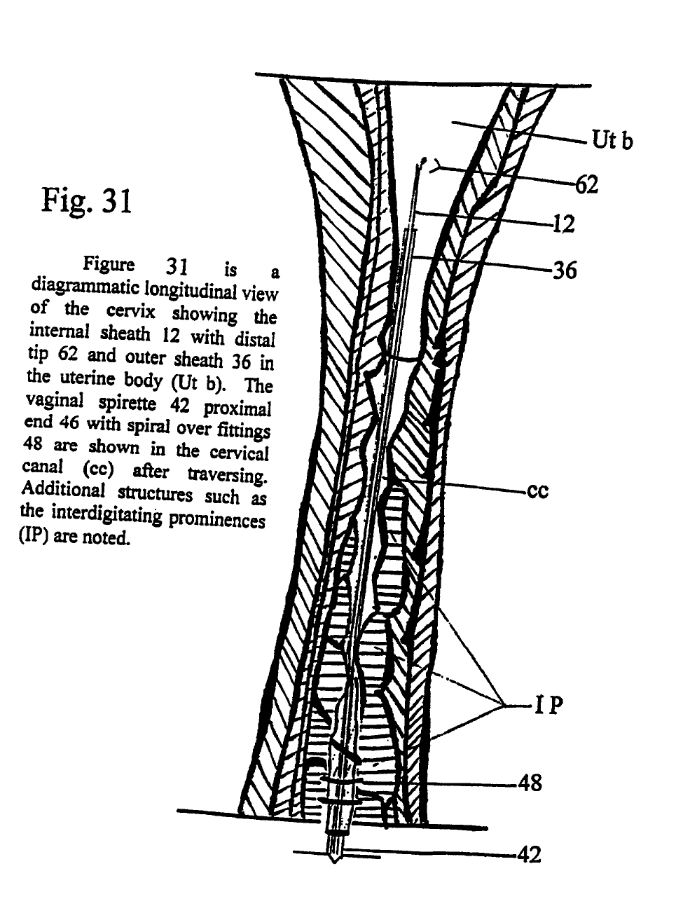

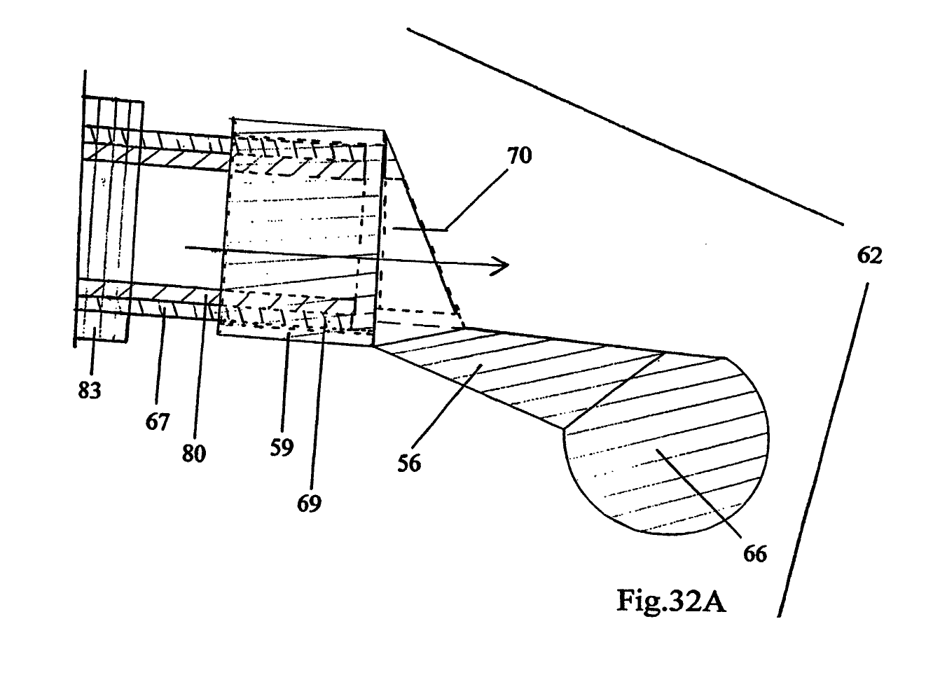

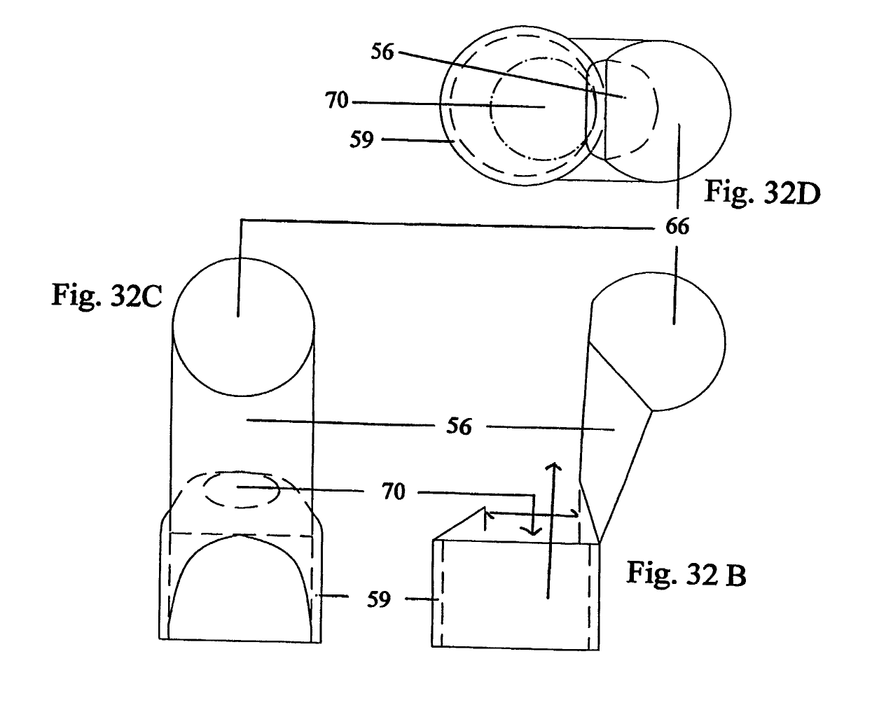

It has been found that using instrument 10 or parts thereof with further steps is also possible for swine embryo transfer. The identical instrument 10 of FIG. 1 or one 10-12 inches longer would be utilized to traverse the cervix using the endoscope and the distal tip 16. The difference from artificial insemination is that once in position, inner sheath 12, with distal tip 16 is withdrawn from the instrument, a distal longitudinal ¼″ slit 39 is also present on the distal tip of outer sheath 36 used in embryo transfers, a second inner sheath 67 (in FIGS. 28-30), preloaded with embryos, is inserted back through outer sheath 36. As shown in FIGS. 29-30 second inner sheath 67 is the same outer diameter (o.d.) of 0.086″ or 2.18 mm as in inner sheath 12 however is longer in length (up to 66 ½ inches) and distal tip 62 in FIGS. 32A-D is similar to distal tip 16, except that a small bore 70 is made through what was window 52 in distal tip 16. An additional innermost plastic sheath 80 containing the embryos is at the distal end of the second inner sheath 67. The internal embryo sheath 80 (FIG. 30) is approximately 11 inches in length, 0.075″ or 1.9 mm o.d. and has a cotton like plug 71 near its proximal end that allows transfer media to be properly placed within sheath 80 to hold the embryos. This cotton like plug stabilizes the transfer media and embryos by not allowing any additional fluid from the uterus to the sheath 80's lumen by pulling the embryos out through the small bore 70 or pushing the embryos towards the proximal sheath 80's end. The distal end of the innermost sheath 80 containing the embryos has a 1-1 ½ inch distal section 81 of the 2^{nd} inner sheath 67 glued to the outer surface of the distal embryo innermost sheath 80. This completed innermost embryo sheath 80 carries the embryos. The innermost embryo sheath section 80 is slid longitudinally inside the distal end of the 2^{nd} inner sheath 69 and pressure fitted or glued thereto, measuring 66 ½ inches in total length, and a completed assembled 2^{nd} inner sheath 67 loaded with embryos. Outside the entire length of the 2^{nd} inner sheath 67 is a 3^{rd} sheath 83, which is approximately 2.8 mm or 0.114″ o.d. in diameter. Traversing the channel of 2^{nd} inner sheath 67 is a small fine diameter wire 84. It has a handle 85 on the proximal end that assists in the directional pushing and pulling of the wire, 75 inches in length. This small diameter wire 84 distal tip is placed against the proximal end of the cotton like plug 71 of the innermost embryo sheath 80 and pushes the plug 71 distally towards distal tip 62 to exit the embryos and transfer media out the exit port 70 to their desired location in the uterine horn. Also, the embryos can be placed into the innermost embryo sheath 80 without the cotton like plug 71. This allows the embryos and transfer media exiting the innermost embryo sheath 80 through the opening 70 by air pressure being injected into the proximal end of the 2^{nd} inner sheath 67 by the syringe 85 thus being placed into the desired location in the uterine horn.

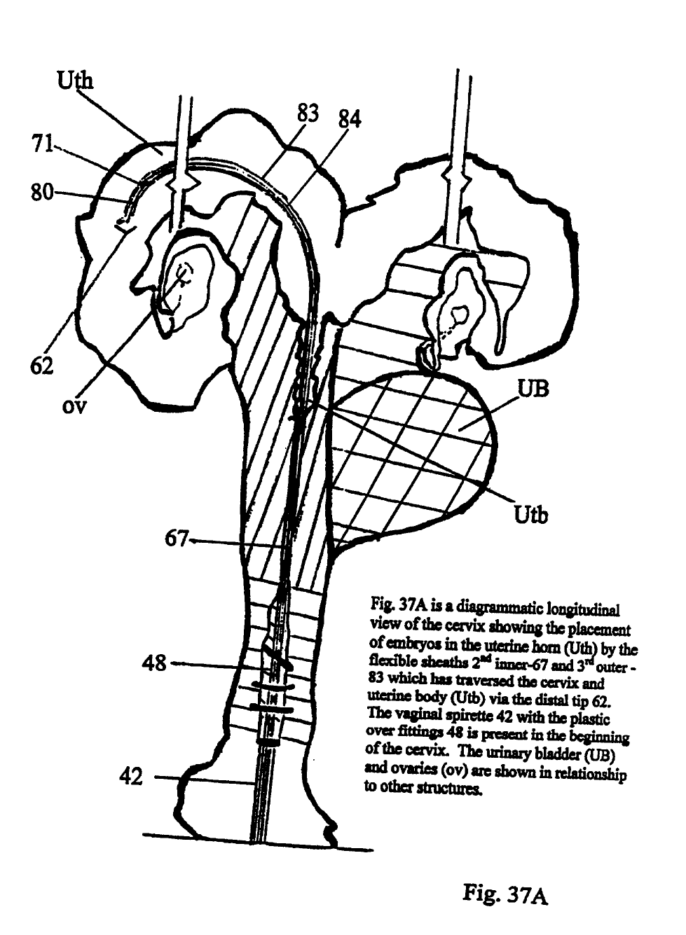

The methodology of embryo transfer would thus be as follows. Spirette or vaginal catheter 42 is placed in the vagina of the sow to the cervix in which the sow will tighten around the vaginal catheter distal end, thereafter cutting the proximal end of the catheter 42 off leaving approximately 12-16 inches in length. The endoscope 20, with the inner and outer sheaths 12 and 36, will be placed within the spirette 42 and directed to the beginning entrance of the cervix. At the cervical entrance, using a counterclockwise rotational movement, using the viewing end of endoscope 29, instrument 10 traverses the cervix and enters the uterine body. Visualization is used to determine identifiable structures or physiological conditions that will eliminate the animal as the possible surrogate female. When the most suitable position is found within the uterine horn, endoscope 29 and 20 with connector 28 are removed from inner sheath 12. Inner sheath 12 with distal tip 16 is then withdrawn from outer sheath 36. The second inner sheath 67 along with 3^{rd} sheath 83, preloaded with embryos in the innermost embryo sheath 80, is placed within the outer sheath 36 and into the uterine body. Once the second inner sheath 67 and 3^{rd} sheath 83 has entered the uterine body, they are navigated by counterclockwise rotation allowing the guide probe tip 66 to assist the navigation of the sheaths 67 and 83 further up the uterine horn to the most desirable embryo placement site as shown in the diagram of FIG. 37A. Embryos are loaded in such a manner that an inch or more of transfer medium is drawn into the innermost embryo sheath 80 drawn up against the cotton like plug 71, next an inch of air space, then a one and one-half inch transfer media with embryos, then an inch of airspace, then two inches of transfer media and an inch or so of air space that completes the embryo loading process. Once this innermost embryo sheath 80 is properly loaded, it is slid into the longer 2^{nd} inner sheath 67 and manually tightened by squeezing the distal end 69 of the 2^{nd} inner sheath 67 or by gluing with a non-embryocidal glue on the outside of the innermost embryo sheath 80 securing it into the distal end 69 of the proximal 2^{nd} inner sheath 67. Placing it in the uterine body, guide probe tip 66 of the embryo loaded 2^{nd} inner sheath 67 now performs the traversal of the uterine body via rotational movement further up the uterine horn to the desired location. If no cotton like plug 71 is present, the embryos are deposited using air pressure applied by syringe 85, attached to the proximal end of second inner sheath 67. If cotton like plug 71 is present in the innermost embryo sheath 80, the embryos are deposited using the fine diameter wire 84 to push the cotton like plug 71 to the distal tip 62 near the opening 70.

This process, traversal of the cervix, uterine body, and the uterine horn to deposit swine embryos, can be accomplished non-surgically and in a non-anesthetized, non-estrus (not in standing heat) standing female animal. This is in contrast to conventional anesthetized, surgically emplaced embryo procedures. It should be noted that surgical emplacement generally means that the female animal will only be able to produce offspring one or two more times because of the damage surgery does to the female.

C. REFRACTORY ARTIFICIAL INSEMINATION

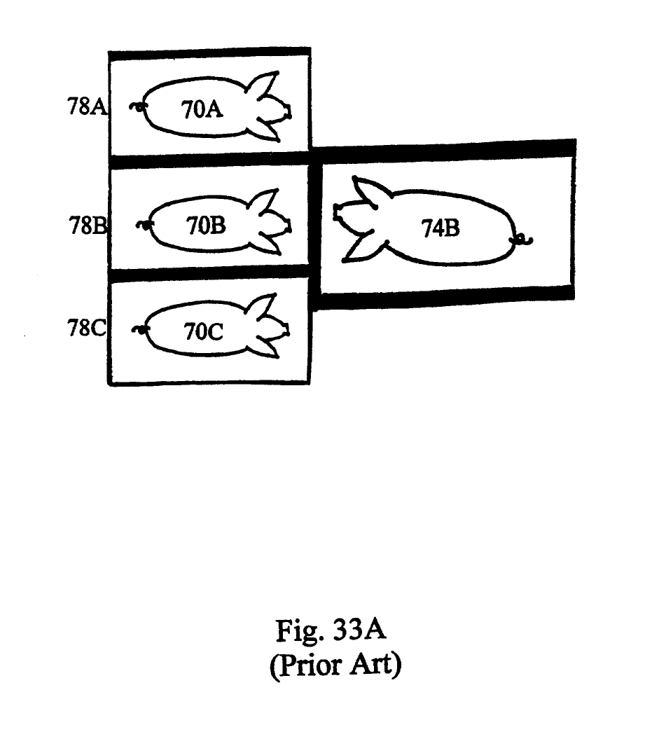

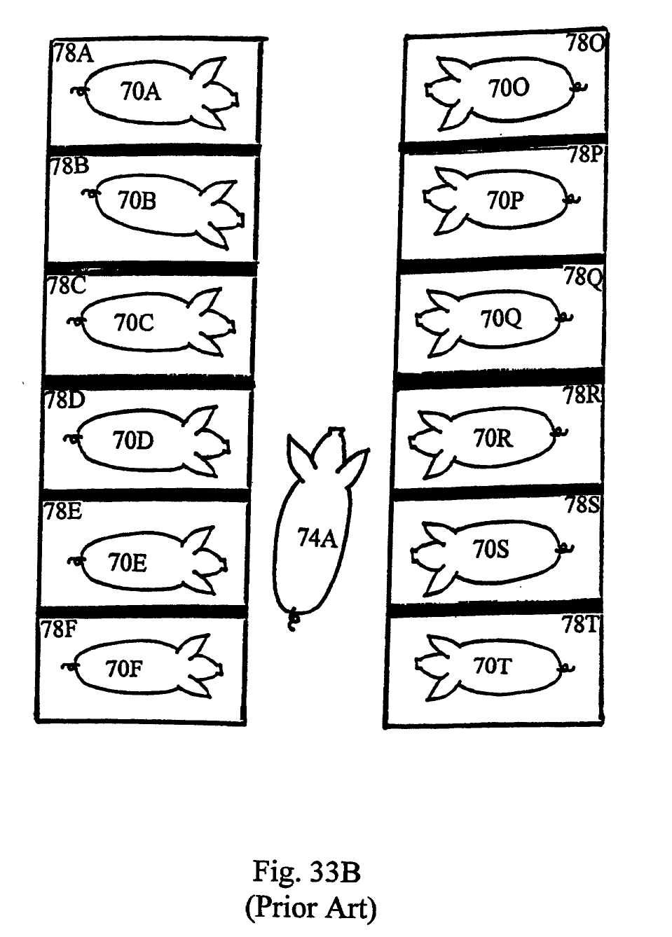

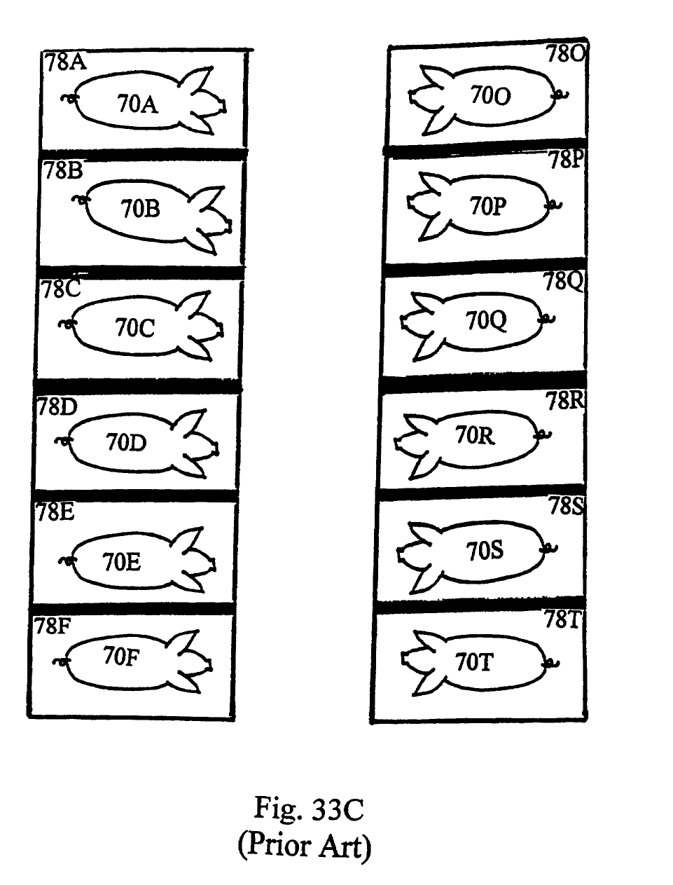

Conventionally, artificial insemination occurs by placing several breeding crates 72 adjacent to a like number of crates 76. The sows 70 are placed in breeding crates 72 nose-to-nose with a like number of boars 74 in crates 76. This close, one-to-one contact between sows 70 and boars 76 induces the biological response in sows 70 to assist in the artificial insemination effectiveness and process. FIG. 33A diagrammatically illustrates a conventional artificial insemination setup in an artificial insemination barn or area. Three breeding crates 72A-C are placed adjacent to three-boar crates 76A-C. Workers gather three boars 74A-C and place them in crates 76A-C. The first three sows to be inseminated (sows 70A-C), are gathered and placed into breeding crates 72A-C nose-to-nose with the boars.

Artificial insemination ensues for sows 70A-C. Once complete, sows 70A-C are removed from crates 72A-C and another set of three sows 70D-F, are placed in crates 72A-C, artificially inseminated, and then removed for subsequent sets.

This process requires the availability of multiple boars as well as the time and effort to corral and maintain the sets of sows during the artificial insemination. It also requires up to three-boar crates 76A-C, which involves floor space. The configuration is basically dedicated to artificial insemination.

FIGS. 33B-C illustrates an artificial insemination process according to the invention. A plurality of sows 70A-70T is placed in confinement structures 78A-78T. These do not necessarily have to be dedicated artificial insemination crates. A boar number 74A is walked by sows 70A-70T to identify which sows are in heat. Artificial insemination by the technique and with instrumentation described earlier can then be used to artificially inseminate sows 70A-T. It can avoid having dedicated artificial insemination space. Container 78A-T might have dual or even more functions and thus further avoid having to bring relatively small sets of sows (e.g. sows 70A-C in FIG. 33A) in for one artificial insemination round, take those sows out and replace them with another group of sows. As indicated in FIGS. 33A-C, a greater number of sows could be ready to be artificially inseminated without such time consuming steps.

Still further, this process saves labor, and time. It also reduces health issues, for example, the possibility of disease transfer that occurs with nose-to-nose, one-to-one, sow/boar processes such as shown in FIG. 33A.

This method, referred to herein as refractory insemination, is believed to be possible with other artificial insemination apparatus and techniques other than disclosed and described herein.

D. OPTIONS AND ALTERNATIVES

It will be appreciated that the foregoing description sets forth exemplary embodiments of apparatus and methods according to the present invention. These exemplary embodiments are given by way of example only and not by way of limitation to the invention. Variations obvious to those skilled in the art will be included within the invention.

It is believed that the apparatus and methods disclosed herein are relevant to other animals beyond swine. Examples would be animals having larger reproductive anatomies or on the same scale as swine. Examples could be elk, deer, cattle, and horses.

Still further, the instrumentation and methodology described above could also used to non-surgically, with no anesthesia, medically treat a reproductive organ of an animal. For example, an infusion of medicine to the uterus could be quickly and atraumatically accomplished to specific sites. The invention allows minimal traumatic traversal for intrauterine treatments.

What is claimed is:

1. An apparatus for artificial insemination of animals comprising: (a) an elongated sheath having an outside diameter, an inside diameter, and an internal passageway between proximal and distal ends; (b) a probe end at the distal end of the elongated sheath, the probe end comprising rounded, non-blunt surfaces and an asymmetrical portion extending outside the perimeter dimensions of the rest of the probe end and the distal portion of said sheath; (c) the external dimensions of the sheath and probe end configured to be relatively small for atraumatic navigation to and through the animal's cervical anatomy.

2. The apparatus of claim 1 wherein the sheath is generally tubular.

3. The apparatus of claim 1 wherein the sheath is somewhat flexible.

4. The apparatus of claim wherein the internal passageway of the sheath has an internal diameter that is maximized.

5. The apparatus of claim 4 wherein the internal diameter is maximized to relative to the outside diameter of the sheath.

6. The apparatus of claim 1 wherein length of the sheath is configured for a species of animal to exceed the distance between the uterus of the animal and the exterior of the animal along the animal's reproductive tract.

7. The apparatus of claim 6 wherein the length allows a portion of the sheath remain external of the animal when the probe end is in the animal's uterus.

8. The apparatus of claim 7 wherein said portion of the sheath includes the proximal end of the sheath.

9. The apparatus of claim 8 wherein the proximal end of the sheath is adapted for manual manipulation by a user, including rotation and longitudinal movement.

10. The apparatus of claim 1 wherein the probe end comprises a generally spherical member extended angularly outward and forwardly.

11. The apparatus of claim 1 wherein the external configuration of the probe end and distal end of the sheath based on the smallest diameter of the animal's cervical canal.

12. The apparatus of claim 11 wherein the largest external cross-sectional diameter of the external configuration is approximately on the same order of size as the smallest diameter of the animal's cervical canal.

13. The apparatus of claim 12 wherein the largest external cross-sectional diameter of the external configuration is on the order of about the same to twice as large as the smallest diameter of the animal's cervical canal.

14. The apparatus of claim 1 further comprising an opening in the distal end in fluid communication with the passageway of the sheath.

15. The apparatus of claim 14 wherein the opening is on the same order of diameter as the inside diameter of the sheath.

16. The apparatus of claim 14 wherein the sheath's proximal end is adapted to receive a dose of semen, and the passageway provides a conduit to move the dose from the proximal end of the sheath to the opening in the probe end.

17. The apparatus of claim 16 further comprising a device to inject the dose of semen under pressure into the sheath.

18. The apparatus of claim 1 further comprising a second elongated sheath having an outside diameter, an inside diameter, and an internal passageway between proximal and distal ends; the inside diameter configured to allow slideable insertion of said sheath.

19. The apparatus of claim 18 wherein the inside diameter and outside diameter are close to one another, such that said sheath and the second sheath frictionally engage and that rotation of one cause rotation of the other, but with sufficient forces, they can slide relative to one another.

20. The apparatus of claim 18 wherein the proximal end is adapted for connection to a source of semen, the inside diameter of the second sheath being larger than the inside diameter of said sheath to provide a larger diameter conduit for semen to reduce turbulence and speed up delivery of semen to the probe end.

21. The apparatus of claim 18 wherein the inside diameter for swine is approximately 0.02 inches to 0.2 inches.

22. The apparatus of claim 1 wherein the length of the second sheath is approximately 2 to 3 feet for swine.

23. The apparatus of claim 1 wherein the length of said sheath is approximately two to three feet for swine.

24. The apparatus of claim 1 further comprising a vaginal catheter having an inside diameter and an outside diameter and a passageway between proximal and distal ends, the inside diameter slideably receiving said sheath and said probe end.

25. The apparatus of claim 24 wherein the inside diameter slideably receives said second sheath.

26. The apparatus of claim 14 further comprising an external member on the vaginal catheter to assist anchoring the vaginal catheter in the animal's vagina.

27. The apparatus of claim 26 wherein the external member is a set of spiral or helical shaped flights.

28. The apparatus of claim 16 wherein the external member is a foam-like material.

29. The apparatus of claim 1 further comprising a strengthening member positioned relative to at least a portion of said sheath.

30. The apparatus of claim 29 wherein the strengthening member is a separate tubular member.

31. The apparatus of claim 29 wherein the strengthening member is an increased thickness of the wall of said sheath.

32. The apparatus of claim 31 wherein the increased thickness of the wall is a separate tubular member over the exterior of said sheath.

33. The apparatus of claim 1 further comprising said sheath being made of one piece.

34. A method of artificial insemination of animals comprising: (a) configuring a probe end comprising rounded, non-blunt surfaces and an asymmetrical portion extending outside the perimeter dimensions of the rest of the probe end and the distal portion of said sheath; (b) configuring a elongated sheath having an outside diameter, an inside diameter, and an internal passageway between proximal and distal ends a probe end at the distal end of the elongated sheath, the probe end; (c) configuring the external dimensions of the sheath and probe end to be relatively small for atraumatic navigation to and through the animal's cervical anatomy; (d) inserting the probe end and sheath into the animal's reproductive tract; (e) navigating atraumatically using rotational and/or longitudinal movement and tactile feedback; (e) traversing the cervix.

35. The method of claim 34 using the probe end and sheath to create a wedging action in the cervix to open up the cervix for entry to the uterus.

36. The method of claim 34 further comprising injecting semen through the sheath and out of the opening in the probe end.

37. The method of claim 34 further comprising inserting the probe and sheath in a second sheath of minimized outside diameter, navigating the combination to and through the cervix, retracting the probe end and said sheath from the second sheath, and injecting semen through the second sheath.

38. The method of claim 37 wherein the inside diameter of the second sheath is greater than the inside diameter of said sheath.

39. The method of claim 34 wherein the probe end is used to lift, move around and traverse the cervical structure.

40. The method of claim 34 wherein the probe end and said sheath are approximately on the order of the same size to twice the size of the smallest part of the path through the cervix.

41. The method of claim 40 wherein the size is on the order of 1.5 mm to 3.0 mm for swine.

42. An apparatus for embryo transfer to livestock comprising: (a) an elongated sheath having an outside diameter, an inside diameter, and an internal passageway between proximal and distal ends; (b) a probe end at the distal end of the elongated sheath, the probe end comprising rounded, non-blunt surfaces and an asymmetrical portion extending outside the perimeter dimensions of the rest of the probe end and the distal portion of said sheath; (c) the external dimensions of the sheath and probe end configured to be relatively small for atraumatic navigation to and through the animal's cervical anatomy.

43. The apparatus of claim 42 wherein the length is a function of the species of animal, at least as long as the distance from external the animal to the end of a uterine horn of the animal.

44. The apparatus of claim 42 wherein the probe end comprises a generally spherical portion extending angularly outward and forward.

45. The apparatus of claim 42 wherein the external configuration of the probe end and said sheath are on the order of size of or no larger than two to three times the smallest diameter of the cervical canal of the animal.

46. The apparatus of claim 42 further comprising a set of embryos loaded into said sheath nearer the probe end than the proximal end of said sheath.

47. The apparatus of claim 46 further comprising a wire adapted for insertion through the passageway of said sheath, for advancing the embryos out of said sheath through an opening in the probe end.

48. The apparatus of claim 46 further comprising a device to exert air pressure in said sheath to advance the embryos out of said sheath through an opening in the probe end.

49. The apparatus of claim 46 further comprising a moveable plug positioned proximally of the embryos in said sheath.

50. The apparatus of claim 46 further comprising transfer medium and/or air pockets positioned in the sheath.

51. The apparatus of claim 42 further comprising an endoscope insertable into said sheath to assist navigation visually.

52. The apparatus of claim 42 further comprising the probe end providing tactile feedback to assist in navigation.

53. The apparatus of claim 42 further comprising the probe end providing audible feedback to assist in navigation.

54. The apparatus of claim 42 further comprising a vaginal catheter through which the probe end and said sheath can slideably pass.

55. The apparatus of claim 42 wherein said sheath comprises a separate distal section having the probe end attached to one end, the interior of which is adapted to hold embryos, the opposite end adapted to fit within the passageway of the other part of said sheath and be fixed in place.

56. The apparatus of claim 55 wherein the distal section is fixed in place in the remainder of said sheath by glue.

57. The apparatus of claim 55 wherein the distal section is fixed in place in the remainder of said sheath by sonic welding.

58. The apparatus of claim 42 further comprising a third sheath slideable over said sheath for increasing rigidity of said sheath along at least a portion of said sheath.

59. A method of embryo transfer in livestock comprising: (a) preloading embryos in a section of a elongated sheath; (b) configuring a leading probe end of the sheath to have rounded, non-blunt surfaces and an asymmetrical portion extending outside the perimeter dimensions of the rest of the probe end and the distal portion of said sheath; (c) inserting the probe end into the reproductive tract of the animal; (d) navigating the cervical structure of the animal; (e) using tactile feedback to navigate; (f) when determined to be in position for embryo transfer, advancing the embryos out of said sheath through an opening in the probe end.

60. The method of claim 59 further comprising preloading the embryos in a separable section of said sheath, and fixing the separable section to the remainder of said sheath prior to insertion into the animal's reproductive tract.

61. The method of claim 59 further comprising advancing the embryos in the sheath by inserting a wire through the proximal end of the sheath and moving a distal end of the wire through the sheath to the proximal side of the embryos, and then continuing to move the wire distally.

62. The method of claim 59 further comprising advancing the embryos in the sheath by introducing fluid pressure into the proximal end of the sheath.

63. A method of artificial insemination comprising: (a) placing a plurality of candidate female artificial insemination animals in the same general location; (b) identifying a set of female candidates for artificial insemination; (c) intrauterine artificially inseminating the set of females without a continued presence of a male animal.

64. The method of claim 63 further comprising moving a male animal past the female candidates to assist in identifying the set of females.

65. The method of claim 63 wherein there is not nose-to-nose contact between female candidates and the male animal.

66. The method of claim 63 wherein there is not confinement of female candidates to individual breeding crates.

67. The method of claim 63 wherein the general location is not a dedicated artificial insemination space.

68. The method of claim 63 further comprising using the method of claim 34 to artificially inseminate selected females.

69. An apparatus for artificial insemination of animals comprising: (a) an area for containment of a plurality of female candidates for artificial insemination; (b) a presentation area for a male animal adjacent to the area of containment of females; (c ) an intra-uterine AI instrument.

70. The apparatus of claim 67 further comprising utilizing the apparatus of claim 1 for artificial insemination of selected females.
